(19) United States
(12) Patent Application Publication
(10) Pub. No.: US 2021/0228276 A1
(43) Pub. Date: Jul. 29, 2021

(54) Medical Platform

(71) Applicant: Crisalix S.A., Lausanne (CH)

(72) Inventors: Jaime García Giraldez, Lausanne (CH); Fabian Wyss, Le Mont (CH)

(21) Appl. No.: 17/050,980

(22) PCT Filed: Apr. 29, 2019
(86) PCT No.: PCT/EP2019/060986

§ 371 Date: Oct. 27, 2020

Related U.S. Application Data
(60) Provisional application No. 62/663,830, filed on Apr. 27, 2018.

(51) Int. Cl. A61B 34/10; G16H 20/40; G16H 20/30; G16H 20/60; G06N 20/00; A61B 90/00

(52) U.S. Cl. CPC A61B 34/10; G16H 20/40; G16H 20/30; G16H 20/60; A61B 2090/365; A61B 90/36; A61B 2034/104; A61B 2034/105; G06N 20/00

(57) ABSTRACT

The present invention is related to the medical field, especially to plastic, aesthetic, cosmetic, reconstructive, and any other procedure dealing with changes or improvements in physical appearance. More particularly, the invention pertains to applications of a community software platform for educating patients about the advantages and potential risks of surgical and non-surgical procedures in the field of cosmetic, plastic, or reconstructive medicine, and for obtaining patient consent. Additionally, the software platform provides enhanced methods for providing patient education and performing post-operation monitoring by generating computer simulations of outcomes and potential complications and providing augmented reality renderings of the simulations.

### FIELD OF THE INVENTION

The present invention relates to a computer implemented medical platform. In particular, it relates to methods for generating 3D anatomical simulations and augmented reality environments for simulating cosmetic or reconstructive medical procedures. It is also related to the field of medical diagnostics, education and compliance. More particularly, it concerns methods of educating prospective patients, obtaining patient consent, and diagnosing post operation complications using realistic representations of cosmetic or reconstructive procedures and their associated risks and complications. Additionally, compliance and post-operation complications diagnosis is made more efficient through automation.

### BACKGROUND OF THE INVENTION

Medical procedures are potentially life-changing events with enormous benefits and risks. Educating patients about the risks and benefits of medical procedures is an essential step in the patient intake process. Patient education is also an important part of the operative and post-operative phases of a procedure with patient knowledge and expectations standing as two cornerstones of patient safety and successful post operation recovery. In addition to keeping patients safe and helping them recover, enhanced platforms for patient education are needed to decrease the amount of unnecessary office visits and complications treatments caused by inaccurate self-diagnosis of procedure complications by patients that were not educated about the recovery process enough to form accurate expectations of how they should look, feel, and progress during recovery.

Despite the importance of patient education and the abundant inefficiencies that result from undereducated patents, conversations between doctors and patients with limited visual aids represents the current state of the art of medical education. Although videos and images of most medical procedures exist somewhere on the Internet, this information is often unreliable and difficult to understand unless viewed in the presence of a medical expert. These visual aids provide an incremental improvement over purely oral methods but fail to deliver the comprehensive, interactive, and personalized experience patients need. Accordingly, there exists a well-established need for curating repositories of medical images, videos, simulations, graphical representations, lectures, descriptions, and other mixed media education materials and presenting the curated materials in an intuitive user interface that allows the patient to explore and interact with the material at his or her own pace.

Augmented reality (AR) is a live direct view of a physical real-world environment whose elements are augmented by computer generated input. Unlike virtual reality (VR) which replaces a physical real world with a simulated one, AR platforms focus on enhancing user perception of real-world experiences by, for example, annotating the pages of a classic literary novel, simulating how a room in a building would collapse during an earthquake, classifying a plant or animal species in real time as it is found in the wild or simulating the results and events of a surgical or non-surgical procedure. Applications of AR are widespread and diverse, but each is based on the underlying concept of receiving real-world sensory input, for example, sound, video, haptics, or location data and adding further digital insights to that information.

Methods of providing a realistic simulation of a real-world experience are especially suited to the medical field. Medical procedures are among the most costly, dangerous, and life changing events in a person's life. Accordingly, it is extremely important for patients and physicians to comprehend the complexity, understand the risks, and predict the results of a medical procedure before it occurs.

In light of the shortcomings of state-of-the-art visual aids, there exists a well-established need for realistic digital 3D simulations of medical procedures. To provide the tools patients need to make a truly informed decision about a potentially life changing procedure, such simulations should be personalized for the individual patient, procedure, doctor, and products used in the procedure. Additionally, the simulations should be interactive to show the changes that will occur to the patient's body during and after the procedure. The simulations should also be interactive so that the patient can visualize physical changes to his or her body from every possible perspective and angle of view. Furthermore, the simulations should provide a comprehensive, step-by-step representation of each action during the procedure so that the patient develops a thorough understanding of the associated risks and potential complications.

The process of obtaining informed patient consent is another essential medical process that needs to be improved. Due to the tremendous impact and expense associated with most medical procedures, obtaining informed patient consent before conducting a procedure is an integral component of regulatory compliance, medical ethics, insurance reimbursement, and limiting physician liability. Despite the fundamental role of patient consent in the medial field, the state-of-the-art process for obtaining patient consent is pen and paper. Most consent forms are long, full of complex legalese and medical jargon, and seldom read or understood by patients.

In light of these shortcomings, there exists a well-established need for a patient consent process that is integrated with patient education so that the patient is actually informed about the procedure he or she is consenting to before providing consent. The consent process should be presented through a user interface within a software application to make the process of giving consent more efficient and flexible to fit patient preferences. Additionally, the patient consent software application should ensure the patient reviews all procedure education materials in an interactive way before consenting to the procedure. The patient consent software application should also save the patient's manifestation of consent, whether it be a physical signature in ink, a digital signature, recording, or some other form, in digital format so it can be accessed at any point in the future by patients, doctors, insurance companies, or any other authorized third party.

Post-operative patient monitoring and follow-up are essential components of successful patient recovery. Throughout the recovery process, it is important to report actual complications to physicians as soon as possible without burdening doctors with benign changes or misdiagnosed routine recovery developments. The vast majority of medical procedures are outpatient procedures meaning most of the recovery process is completed at home by the patient with only a few periodic check-ups. Accordingly, most of the responsibility for accurately diagnosing procedure complications falls on the patient who in most cases is not a medical expert and typically has little to no experience recovering from their particular procedure. To make matters worse, there are few technology-based tools for helping patients diagnose complications and monitor their recovery process. As a result, many harmful complications go undiagnosed and many routine recovery symptoms are falsely diagnosed. Both of these problems add significant cost to already expensive procedures while also reducing the efficiency of doctors and other healthcare providers.

Accordingly, there exists a well-established need for automated diagnostic tools that can help patients diagnose complications during the recovery process. There also exists a need for a patient follow up and monitoring software application that can automatically track patient recovery progress and schedule an emergency appointment with a doctor if the patient reports symptoms that carry a high risk of being associated with complications or submits photos of the procedure area that suggest infection or another complication.

Patient education, consent, and post operation monitoring and follow-up are three important but severely outdated medical processes that need to be improved in order to help doctors better care for their patients, to help patients recover faster, and help medical insurance companies and healthcare provides reduce the cost of medical care. Regarding patient education, there exists a well-established need for more realistic procedure education materials that are presented in a more interactive way. For patients without medical education, the process of learning about a new medical procedure should be intuitive, highly visual, and specific to the patient. Such an education experience would allow the patient to develop a keen, personal understanding of the procedure their body is about to endure along with an accurate set of expectations for how recovery should go as well as a clear list of action items to pursue if complications arise. The patient consent process should be integrated into the patient education materials so that patient consent is obtained only after the patient has clearly understood and interacted with the education materials presented to them. Finally, the post operation monitoring and follow-up process should offer more support to patients in the form of automated diagnostic tools that can help diagnose complications and recovery process simulations and reports that provide an accurate idea of what the patient should expect at each stage of the recovery process.

### SUMMARY OF THE INVENTION

In one aspect, the invention provides a computer-implemented method of simulating the effect on a patient's body of a procedure, comprising: receiving a selection of a procedure; creating a pre-procedure 3D model of at least a part of the patient's body that would be affected by the procedure; simulating the effects of the procedure on the patient's body and generating a plurality of post-procedure 3D models from the pre-procedure 3D model, each post-procedure 3D model representing the patient's body at a different time following the procedure; and displaying any of the pre-procedure 3D model and the post-procedure 3D models over a still image or a video of the patient.

In this way, embodiments of the present invention enable the creation of time-based representations of the outcomes of a medical procedure such as a cosmetic or reconstructive procedure, or of the changes over time to a patient's body as a result of implementing a diet or a physical fitness plan. Among many other advantages, this enables education of a patient, for obtaining informed consent and for managing expectations. By simulating outcomes using the patient's body as a base model, the patient can much more easily see and understand those outcomes in order to make an informed decision.

In one embodiment, the method further comprises: receiving a selection of a potential complication of the procedure; and simulating the effects of the complication on the patient's body and generating a plurality of post-complication 3D models from either the pre-procedure 3D model or a post-procedure 3D model, each post-complication 3D model representing the patient's body at a different time following the complication.

Understanding potential complications of a procedure is an important part of obtaining informed consent. Furthermore, by simulating complications using the patient's body as a base model, the patient will understand better what symptoms to look out for and will be able to report potential complications to a physician in a timely manner.

In one embodiment, the method further comprises training a machine learning system on a training dataset comprising a plurality of 3D models of at least parts of a plurality of patients' bodies at different times following a procedure and using the machine learning system to simulate the effects of the procedure on the patient's body.

The use of machine learning or artificial intelligence for simulation purposes means that simulated outcomes, including complications, are based on real-world results and not just on designed templates or mathematical formulas. Simulations based on real results are better able to educate patients and physicians. Following completion of the procedure, 3D models of the actual outcomes can be created from the patient's body and these models can be added to the training dataset of the machine learning system to further improve its simulations.

In one embodiment, the post-procedure 3D models include a model representing the patient's body immediately after the procedure is completed, and at least one model representing the patient's body at a selected time during the procedure. As well as informing a patient, simulated models of instances during the procedure, particularly a surgical procedure, can educate a physician to enable them to perform better.

In another aspect, the invention provides a computer-implemented method of simulating the effects of a medical procedure on a patient's body comprising: training a machine learning system on training data comprising the effects of the medical procedure on a plurality of patients' bodies, as performed by a plurality of different physicians, to generate a plurality of predictive models; creating a 3D model of at least a part of the patient's body that would be affected by the procedure; using a first predictive model, generating a first modified 3D model of the at least part of the patient's body following the procedure, simulating the effects of the procedure as performed by a first physician; and using a second predictive model, generating a second modified 3D model of the at least part of the patient's body following the procedure, simulating the effects of the procedure as performed by a second physician.

The use of artificial intelligence to gain real world data about the performance of multiple different physicians enables the creation of both general purpose models to create generic simulations of the effects of a medical procedure, but also more specific models for creating physician specific simulations. This can provide invaluable insight, enabling the provision of a virtual second opinion on the effects of a medical procedure.

In another aspect, the invention provides a computer-implemented method of obtaining patient consent for a medical procedure comprising: receiving a selection of a medical procedure; receiving patient information including at least a patient location; automatically determining consent requirements of the patient based on the patient location and the medical procedure and retrieving at least one consent workflow meeting the consent requirements from a store of consent workflows; automatically identifying at least one education course needed to educate the patient about the medical procedure and retrieving the at least one education course from a store of education courses; using the or each retrieved consent workflow and the or each retrieved education course, automatically assembling an education and consent workflow for educating the patient about the medical procedure and for capturing patient consent to the medical procedure; displaying the education and consent workflow; receiving affirmation of consent from the patient; and storing the education and consent workflow and the affirmation of consent.

The computer-automated assembly of an education and consent workflow ensures that necessary laws in the jurisdiction in question can be complied with. Furthermore, assembling the consent requirements together with education courses and providing these together over a computer platform provides the patient has the best possible understanding of the procedure and what they are consenting to, while storing their affirmation of consent together with the education and consent workflow offers protection to the physician. On a digital platform, the affirmation of consent may even include video of the physician talking through the education course with the patient to demonstrate informed consent providing further legal protection for the physician.

In one embodiment, the patient information includes at least one image of the patient's body; and assembling an education and consent workflow comprises automatically simulating at least one outcome of the medical procedure using the or each image of the patient's body to create a simulated representation of the at least one outcome on the patient's body, and including the simulated representation in the education and consent workflow. Personalising an education course using simulated representations of outcomes on the patient's actual body increases the patent's understanding and better informs consent.

In another aspect, the invention provides a computer-implemented method of diagnosing patient complications during recovery from a medical procedure comprising: receiving patient recovery data via a patient device; extracting, by a data analytics service, patient recovery parameters from the patient recovery data; ingesting, by a diagnostic AI, the patient recovery parameters in order to identify procedure complications within the patient recovery data based on the extracted patient recovery parameters; producing, by the diagnostic AI, a complications diagnosis; assembling, by a complications application, a complication report including the complications diagnosis and a treatment plan; and delivering the complication report to the patient device.

Using an artificial intelligence system to diagnose complications from a medical procedure enables early assessment of potential complications or can set the patient's mind at ease if the diagnosis is clear. For more serious complications, the complication report can be passed on to a physician for human review, and the computer system can optionally automatically schedule an appointment for the patient.

In another aspect, the invention provides a computer-implemented method of generating an augmented reality (AR) rendering of a medical procedure, comprising: receiving a selection of a medical procedure affecting a body part of a patient, patient measurements, and an image of the body part of the patient; generating, by a 3D modelling engine, a 3D model of the body part comprising a three-dimensional mesh structure covered in a texture material, the patient mesh structure dimensioned according to patient measurements, and the texture material extracted from the image of the body part; simulating, by a simulation engine, modifications to the 3D model according to an anticipated result of the medical procedure; and matching, by an AR engine, the position and orientation of the 3D model with the position and orientation of the body part of the patient in a video of the patient, and augmenting the video with a rendering of the modified 3D model over the body part of the patient.

Advantageously, preparing a 3D model of the patient's body, textured with an image of the patient's body, and then modifying that 3D model based on the likely result of a medical procedure, enables the patient to easily understand how the procedure will affect them. Furthermore, by matching the modified 3D model to a video (including a live video) of the patient's body using an augmented reality system, the patient is immediately and intuitively able to observe the overall effects of the procedure on their body.

In general, aspects and embodiments of the present invention include a software application for improving three core aspects of the patient medical experience—patient education, patient consent, and post-operative patient monitoring and follow-up. The patient consent and post-operative monitoring and follow-up aspects are integrated with the education aspect to provide a cohesive user experience that delivers visual, intuitive, interactive, and realistic education materials at the appropriate time in the pre-operative and post-operative stages of any surgical or non-surgical medical procedure. For example, the process of obtaining patient consent may be a multipart process that is included in a procedure education course. At the end of each lesson in the education course, there may be a prompt to consent to the specific aspect of the procedure that was just reviewed in the lesson. Consent may be obtained through the software application in a variety of ways including downloading a consent form and signing in ink, checking a box, signing digitally or some other outward digital action that manifests consent, or recording a video or audio message that includes the patients name and an expression of their intent to consent to the procedure.

The software application may integrate with a camera application and microphone application on a patient or physician device to process the recording. In this example, the device camera records a video of the patient while the device microphone records audio of the patient stating the agree to all terms of the procedure and they he or she understands the procedure and all of its potential complications and risks. Optionally, the consent aspect of the application may present the patient with a questionnaire containing predefined questions that elicit patient consent to a particular procedure. The questionnaire could be formatted as a recording or a written document. In the recording example, a device speaker would play a recording asking the question and the patient would respond by speaking into a device microphone. In the written format, the patient would check a box, click a button, or otherwise signal their affirmative or negative response to each question. In both cases to responses submitted by the patient are saved on the device and the content of the questionnaire can be modified according to the patient, procedure, physician, doctor's office, patient's insurance, or legal requirements of the state, country, or geographic jurisdiction having the authority to govern the procedure. The consent aspect of the application be used in the presence of a physician or accessed by the patient remotely outside of a consultation with a physician to avoid intended or unintended influence on the patient by the physician.

The consent aspect of the application may also contain multiple styles of consent form with the content included in of each version of the consent form corresponding to the patient consent requirements of a particular state, country, city, or other geographically dependent legal jurisdiction. The consent aspect may also include an automated means for selecting the consent form to use according to the patient's nationality, the location of the clinic or office performing the procedure, or some other geographic indicator of the jurisdiction governing the procedure.

The education and post-operative patient monitoring and follow-up aspects of the software application may include an augmented reality (AR) platform for rendering 2D/3D models and simulations of medical procedures and post-operative complications to allow patients and physicians to assess the potential physical changes to the patient's body that may occur as a result of a successful procedure or complications that occur during recovery.

In one embodiment, the platform includes a computer system that provides patient models and procedure simulations that display the effect of each step of a procedure on the patient's own body. For example, the computer system provides a procedure simulation by comparing a pre-operative 3D model generated before the procedure to one or more 3D post-operative models. In this example, the pre-operative 3D model depicts the patient's body before the procedure and is generated from photos or videos of the patient's body. One or more post-operative models are then generated by the computer system based on a set of input parameters such as patient demographics, type of procedure, desired simulation time intervals, physician performing the procedure, and the products and/or product brands used in the procedure. These input parameters may be selected manually or be automatically detected using an artificial intelligence system. The post-operative models depict changes to the patient's body that occur as a result of the procedure. In one example, the changes are shown through a series of post-operative models depicting one or more intermediate steps concluding with a depiction of the patient's body when they have fully recovered from the procedure. Alternatively, the post-operative models may depict changes to the patient's body that occur as a result of actions by the physician during the procedure. One example simulation includes a series of four post-operative models for a breast augmentation. The first model depicts the patient's body after receiving anaesthetic, the second model shows the physician making an incision, the third model displays the physician inserting the implants, and the fourth model shows the physician suturing the incision sites.

The location and effect of any incisions, injections, substance removal, application of a product, suturing, or any other physical manipulation made by the doctor during the procedure will be visualized on a representation of the patient's actual body. These models and simulation allow the patient and physician to visualize the effect each step of the procedure will have on the patient's body in advance of the performing the procedure.

In one example the AR system of the present invention generates 3D models of patient bodies from 2D images and/or 3D body scans. The AR system then compiles one or more generated 3D models into a procedure simulation that includes additional virtual representations of the effects of physician actions during a procedure. To produce a simulation that can be viewed over a patient's physical body in real time, the AR system automatically aligns, positions, renders, completes, and buffers one or more 3D models and virtual representations of the effects of physician actions on the models to generate an interactive simulation that shows changes to the patient's body during a procedure.

The AR system further provides a graphical environment that displays a 360° representation of the 3D model and corresponding procedure simulation. The 3D model and simulation are presented in an interactive display that allows users to rotate and angle the model and/or simulation to view a complete range of perspectives and viewpoints. In one example, the interactive display supports touch screen and/or click through user inputs that allow users to rotate, angle, and otherwise change the perspective of the 3D model and simulation by touching or clicking on the model or simulation and dragging the virtual representation to a desired position or perspective. In an alternative example, the 3D model and simulation are presented in an augmented reality environment that projects the changes to a patient's body onto a live video of the patient in real time. In this example, users can change the position of the 3D model or simulation by moving his or her physical body. The system automatically detects the body part to be augmented, projects a virtual image of the body part with the effects of the procedure onto the actual body part, tracks the actual body part in real time, and changes the angle and perspective of the projected virtual image of the changed body part according to real time changes in the position of the actual body part.

Another aspect of the AR platform included in e-learning platform is a 2D/3D model editing tool that integrates simulation of anatomical aesthetics produced by a procedure into the education process. In one example, the tool generates a 2D/3D model of at least one body part or anatomical region. The 2D/3D model is then visualized in a virtual reality (VR) or augmented reality (AR) environment or as a 3D model on a 2D screen. When visualized, the 2D/3D model may be manipulated within the VR/AR environment by rotating the 2D/3D model up to 360 degrees on multiple axes.

Within the VR/AR environment or on a 2D screen display the 2D/3D model may rotate around vertical axis running through the horizontal centre of the 2D/3D model. Rotation through the vertical axis creates a first visual effect of spinning the 2D/3D model around a fixed vertical point so that all side surfaces of the 2D/3D model are visible. The 2D/3D module may also be rotated 360 degrees around a horizontal axis running through the vertical centre of the 2D/3D. Rotation around the horizontal axis creates a second visual effect of spinning the 2D/3D model around a fixed horizontal point so that the top and bottom surfaces of the 2D/3D model (e.g., in a 2D/3D model of a head and face the top of the head and underneath the nose and chin) are visible. A zoom feature enabling magnification and demagnification of selected features of the 2D/3D model may also be used to magnify certain aspects of the model.

The 2D/3D model editing tool may be compatible with a touchscreen and/or pen or stylus to enable rapid, intuitive, and precise editing. In one example, to edit a 2D/3D model to show a patient the anatomical aesthetics that could be produced by a successful procedure, a finger, pen, or stylus can be used to draw one or more boundary lines (i.e. lines defining dimensions) of one or more anatomical features. The AR/VR and/or 2D screen model display provided by the e-learning platform may automatically adjust the 2D/3D model to reflect the new dimensions for one or more anatomical features defined by the drawn boundary lines.

In one example, if the boundary lines are drawn inside the 2D/3D model the VR/AR environment or 2D display may shrink one or more anatomical features so that the features do not extend beyond the drawn boundary line. In another example, if the boundary lines are drawn outside the 2D/3D model the VR/AR environment or 2D display may enlarge the one or more anatomical features to extend the features out to the boundary lines. A free draw setting of the 2D/3D model editing tool drawing feature may be responsive to the exact movements of a finger, pen, or stylus on a touchscreen to enable drawing curved, angled, straight, or some combination of features within a single boundary line. Alternatively, the drawing feature may have one or more assisted draw settings that lock boundary line dimensions so that they are straight, maintain a certain shape, and/or are proportional to existing anatomical features.

The simulation aspect of the AR platform included in an e-learning platform may be coordinated with the 2D/3D model editing tool to allow real time simulation of models edited using the 2D/3D model editing tool. In one example, the simulation aspect has one or more sliders or other simulation timing mechanisms that display process of the simulation from the current unadjusted model to the post procedure model and/or from the original post procedure model to the edited post procedure model. In one non-limiting example, a toggle button within the slider may be moved manually to show a specific model simulation position between an original model and an edited model. Alternatively, the slider may be synchronized with a timer that gradually adjusts the original model to the edited model over a defined time interval (e.g. 10 s, 15 s, or 30 s).

The simulation aspect may also allow patients to see themselves in a mobile device selfie mode. In one embodiment, patients can use a selfie mode of the AR platform to see their actual face and then simulate the effects of aesthetic products using an image filter that overlays a simulation over an actual image or video of a patient. The image filter may simulate a filler and/or botox procedure that reduces wrinkles and/or folds in the skin. In other embodiments, the image filter may simulate a lip injection increasing the volume of a patient's lips. Image filters may also simulate modifications to other body parts including breast implants and/or fat reduction procedures. Image filters may be specific to a particular procedure or product. For product specific image filters, many image filters may exist for the same procedure, wherein each image filter simulates effects specific to a particular product.

The 3D models of the present invention can be generated using a number of photos from the patient or any external 3D scanning device. Patients can generate 3D models themselves at home or consult a physician for assistance. The generation of the 3D models can be done by the physician or by the patient itself. The system described herein provides several methods of comparison between the pre-operative models and post-operative models achieved using different product brands, physicians, or surgical or non-surgical techniques. The methods of comparison provided by the AR system described herein include a side by side comparison of static or dynamic 3D models as well as a 3D simulation that displays an incremental transition from the pre-operative model to the post-operative model over a defined time interval. In addition to displaying 3D models and simulations, the AR system may also generate one or more quantitative metrics to describe the transition of a patient's body from a pre-operative state to a post-operative state. In a preferred embodiment, the quantitative metrics include point-to-point distance, over the surface distance, and volumetric measurements. The quantitative metrics for the pre-operative and post-operative models may be manually defined by the physician or patient during a consultation or remotely. Alternatively, the quantitative metrics may be automatically generated using one or more machine learning algorithms or artificial intelligence systems trained on patient and physician data specific to the particular simulated procedure.

In one example, a patient or physician would use the AR system to visualize how the patient's body will look before the procedure relative to how the same patient body will look after the procedure. Displaying the post-operative effects on the patient's body provides the patients with an intuitive understanding of the risks and potential complications associated with the procedure as well as an idea of how long it may take to recover from the procedure. In addition to displaying 3D models and simulations the AR system may also generate a recovery time or difficulty prediction based on the characteristics of the patient, for example, recovery environment, health, age, and other demographic information, the type of procedure endured by the patient, the doctor who performed the procedure, and the product brands or materials used in the procedure. By seeing realistic changes to his or her body that occur as a result of the procedure and viewing the recovery predictions, the patient can make a more informed decision about whether the advantages of the procedure are worth the cost and potential risk of complication. Additionally, by modelling and simulating the physician actions necessary to complete the procedure on a realistic patient body representation, the physician can better visualize the physical mechanics of performing the procedure and optimize where, when, and how to perform each step of the procedure to minimize the risk of complications. In turn, the patient can better understand the procedure he or she is about to undergo generally as well as the specific steps of the procedure that pose the greatest risk to his or her health and safety.

Integrating this simulation into the education and consent aspects of the software application provides the patient with an intuitive, visual understanding of the effect the procedure will have on his or her body to better inform their decision to consent to undergo the procedure. Providing the simulation in advance of the procedure also allows the patient an opportunity to ask the doctor about any aspects of the procedure they do not understand before decided to give consent. Additionally, doctors can use the simulation as a training tool for identifying challenging steps of the procedure or patients that are more likely to experience certain complications or side effects after undergoing a particular procedure. In one example, the simulation is generated using actual 2D images and/or 3D scans of the patient's body as well as digital representations of the steps performed by a physician in a particular surgical or non-surgical procedure. Generic patient models and procedure simulations can be augmented according to a set of input parameters including patient demographics, type of procedure, desired simulation time, time intervals between each procedure step, physician performing the procedure, and the products and/or product brands used in the procedure. These input parameters may be selected manually or automatically detected using an artificial intelligence system.

Alternatively, the education and consent aspects of the software system may be augmented by AR based 3D models of patients before and after a procedure with education content including text, images, slides, videos, audio, and other mixed media content relevant to the procedure the patient is investigating. The software application may have a user interface that requires the patient view, acknowledge, or otherwise interact with the education content presented by the software application in order to reach the consent aspect of the application. In this example, the software application presents a consent form to the patient only after the patient has viewed all of the education content relevant to the particular procedure he or she is providing consent to undergo. In this way, the software application prevents patients from consenting to procedures they do not fully understand, thereby providing a solution to the problem of patients signing consent forms they have not fully read.

AR based patient recovery models and simulations can be incorporated into the education and post-operative patient monitoring and follow-up aspect of the software application to provide a more realistic view of the recovery progress. The post-operative models depict changes to the patient's body that occur as a result of complications with a particular procedure. In one example, the deterioration of the patient's body overtime as a result of the procedure complications is shown through a series of post-operative models depicting one or stages of infection, deterioration, or other complications, for example, a series of four post-operative models may depict the patient's body after a complication is just starting to show in the first model, after 3 days of no treatment in the second model, after a week of no treatment in the third model, and after a month of no treatment in the fourth model. Simulations of post-operative complications may be provided to the patient during the pre-operative phase to give a better understanding of the risks and potential complications associated with the procedure. Alternatively, simulations of post-operative complications may be provided to the patient during the post-operative recovery phase.

To provide more realistic representations, post-operative complication simulations presented to patients after a procedure may be generated from 2D images or 3D body scans of the patient's body after the procedure. These post-operative models display complications directly on the patient's body as it looks after the procedure to enhance patient understanding of how to diagnose complications. The post-operative models may be augmented with patient specific information including patient demographics, type of procedure, desired simulation time, time intervals between each complication phase, physician performing the procedure, the success of the procedure and the products and/or product brands used in the procedure.

The software application described herein further provides a messaging platform for distributing the generated 3D models and simulations. Patients can use this messaging platform to share 3D models and simulations of their body through text message or email as well as in a web-based chat or social media application. Physicians can leverage the messaging platform to share 3D models and simulations of prospective patients with other physicians and medical professionals to get a second opinion or a complex case, receive product or brand recommendations, or refer a patient to another physician, practice group, or medical office.

The patient follow-up and consent aspect of the software application further includes one or more machine learning models or artificial intelligence systems for diagnosing procedure complications from 2D images and/or 3D body scans provided of a patient's post-operative body. The diagnosis may be based on automated image classification results informed by real world diagnostic methodology from surgeons. In one example implementation, the artificial intelligence system aggregates images of body parts having infections, deterioration, or other complications. These images may be sourced from a third party, for example, a data provider or medical research institution, or provided by the physician personally, the physician's practice group, clinic, office, or hospital system. The images are tagged for the complications pictured and optionally augmented with additional information for one or more internal and/or external data sourced, for example, patient, physician, practice group, doctor's office, procedure, product, and or product brand information relevant to complications for a particular procedure or class of procedures. This image and textual data may be collected using manual and/or automated methods and may be periodically updated through manual or automated methods.

The artificial intelligence system ingests the tagged image data and may associate it with one or more text fields, for example, the age of the patient, the brand of material used, or the doctor performing the procedure. The system may use the associations, raw image data, or some combination to classify the data into one or more types or archetypes of data. The artificial intelligence system then selects a training data set from the raw or classified data. The artificial intelligence system then trains one or more artificial intelligence models or algorithms using the training data. From this process, the artificial intelligence system produces one or more artificial intelligence models or algorithms that encompass insights related to diagnosing complications from one or more medical procedures that are machine learned from the training data set. The one or more artificial intelligence models or algorithms provided by the artificial intelligence system may be ensembled into a convolutional neural net that makes predictions based on pixel positions in the image provided by the patient relative to images included in the model's training set. Alternatively, the models may be interferenced individually or arranged in different multi-model that makes diagnostic predictions based on a comparison of the images provided by the patient to images of other patients with complications included in the training set. Additionally, procedure specific or complication specific models may be trained using only images of particular procedures or complications. The models are used to diagnose the existence of complications in patients recovering from surgical or non-surgical procedures based on real world data collected from actual procedures conducted on real patients. The artificial intelligence system may also be trained to recognize physiological anomalies specific to one patient using a model trained on pre- and post-operative images from that one patient's body. Complication diagnoses made by the artificial intelligence system may be validated by a physician and incorporated into the training data to improve model accuracy.

The input data for the artificial intelligence system includes all post-operative images and 3D models provided by every physician and patient connected to the system. As new models are produced, the training dataset of the artificial intelligence system may be updated with the new data in real time to continuously improve model and simulation accuracy. Information aggregated by the artificial intelligence system may include data provided by physicians about the surgical or non-surgical procedure including the type of procedure, any products used to perform the procedure, product specifications, and the surgical or non-surgical techniques used. The artificial intelligence system may also aggregate patient data including physical information such as weight, height, and age as well as any other information that may help the system provide more accurate diagnostic predictions.

The artificial intelligence system can be used in a generic way to encompass data aggregated from all physicians using the technology. These generic diagnostic models are the most generalizable because they include data on all available post-operative images and 3D models. Alternatively, more specific models or algorithms may be trained on only the data for one physician, practice group, or doctor's office. These more specific models are less generalizable but may be more accurate for patients recovering from procedures performed by the specific doctor or practice group because the model is tailored to the individual characteristics, experience, and results of a single physician or group of physicians. Physicians may select between the generic system, their own personalized models, or the models based on other physician's results to provide a better complications diagnostic model for their patents and compare diagnostic predictions across different physicians performing the same procedure. Patients may also access the system remotely and select between the generic or specific models to more accurately diagnose complications as well as compare diagnostic results between different physicians or groups of physicians. In this way, the computer system described herein provides patients a virtual second, or third, or fourth, and so on opinion. Accordingly, patients may use the invention to diagnose potential complications in advance of scheduling a consultation. The artificial intelligence system may also include a recommendation system for providing patients the physician or group of physicians having the most favourable post-operative results and complications diagnostic data for a particular procedure performed on a person with the patient's particular individual characteristics or similar characteristics.

The artificial intelligence system may also be used to predict post-operative complications according to different medical products or product brands. The product specific models provide patients' and physicians' complications diagnoses specific to a particular product or product brand. This feature can be used to balance the cost of a more expensive product against the likelihood of experiencing complications and the severity of the complications typically observed in patients using premium products in procedures relative to less expensive alternatives. Accordingly, patients and physicians can use the artificial intelligence system to select the products and product brands to use in a particular procedure. The artificial intelligence system may also include a recommendation system that suggests particular products or product brands that achieved the best post-operative results for a particular procedure performed by a particular doctor on a patient having the same or similar characteristics to the patient being evaluated.

Other specific models generated by the artificial intelligence system include models specific to a particular geographic region or demographic, for example, a particular age group, occupation, socio-economic status, or ethnicity. The artificial intelligence system can also be trained to diagnose complications at various stages of recovery including, for example, complications after 24 hours, 2 days, 5 days, one week, two weeks, one month, three months, or six months.

### BRIEF DESCRIPTION OF THE DRAWINGS

FIG. 1 illustrates a client-server environment of a computer system that provides the patient education, consent, and follow-up application.

FIG. 2 illustrates the server-side components included in an example server environment of a computer system for providing functionality to the patient education, consent, and follow-up application.

FIG. 3 displays an example workflow for generating an automated complications diagnosis.

FIG. 4 illustrates a data ingestion pipeline for providing data to an AI system for diagnosing post-operative complications.

FIG. 5 shows an example workflow for obtaining informed patient consent using the patient education, consent, and follow-up application.

FIG. 6 displays an example home screen of a user interface for interacting with the patient education, consent, and follow-up application.

FIG. 7 displays example complication images provided by the patient education, consent, and follow-up application.

FIG. 8 displays example text content provided by the patient education, consent, and follow-up application.

FIG. 9 shows an example patient consent intake modal generated by the patient education, consent, and follow-up application.

FIG. 10 shows an example imagine engine.

### DETAILED DESCRIPTION OF THE INVENTION

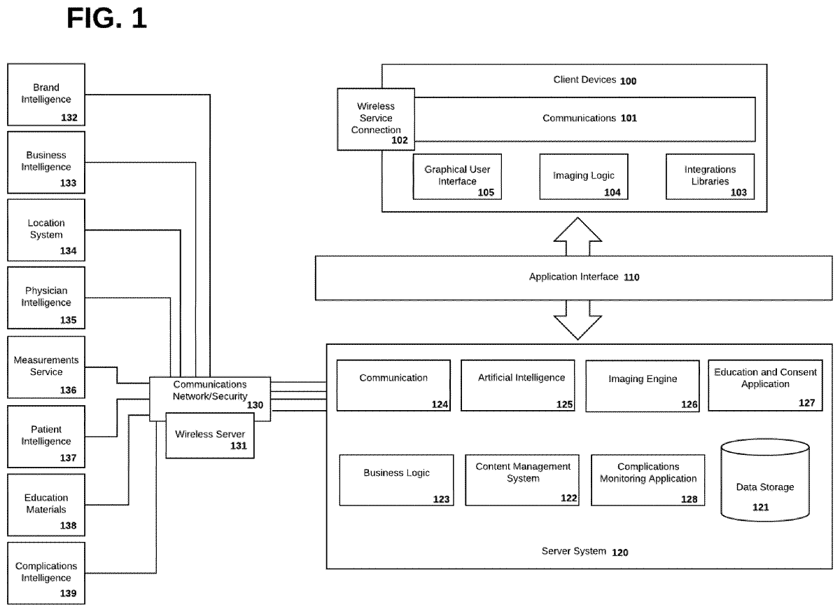

FIG. 1 illustrates a client server arrangement of the patient education, consent, and follow-up application. This arrangement provides functionality of the patient education, consent, and follow-up application including education content, patient consent workflows, post-operative follow-up questionnaires, automated complications diagnosis, 3D models, procedure simulations, and AR environments to patients and physicians in an interactive user interface. The arrangement includes one or more client devices 100 that interact with one or more server system 120 components through an application interface 110, for example an application programming interface (API) written in a programming language, for example, PHP, Python, Java, Node, or JavaScript.

The client device 100 components are implemented in a web based or mobile application programmed to run on a plurality of computing devices for example, a desktop computer, laptop, tablet, mobile phone, or smart phone. The client device 100 components include a communications module 101 that provides a wireless service connection 102 for interfacing with the server system 120 components, one or more internal or third-party services or computer systems, for example, 130-139, or other applications connected to the Internet. Information from received from the wireless service connection 102 is provided to the graphical user interface (GUI) 105 for display and further processing. The imaging engine 104 generates 3D models, simulations, and AR environments that provide realistic representations of surgical and non-surgical procedures as well as post-operative results and complications associated with such procedures.

The imaging engine 104 interfaces with the AR system 126 component of the server system 120 through one or more integrations libraries 103. The integrations libraries may include one or more rendering libraries to compile, arrange, and/or buffer one or more models generated by the imagine engine into a static or dynamic simulation. In one example, the dynamic simulation is a static representation of the post-operative results of a surgical or non-surgical procedure. Another example includes a transformational simulation depicting every step of a surgical or non-surgical procedure. One example transformational simulation of a breast augmentation surgery may include transitions between four 3D models with the first model depicting the patient's body receiving anaesthetic, the second model showing incisions made on the patient's body, the third model displaying implants inserted into the patient's body, and the fourth model showing the patient's body with sutured incision sites. Other example transformational simulations include progression of an infection, implant material deterioration, body deterioration, or other complication over a defined time interval such as 3 days, a week, or a month.

Models provided by the imagine engine 104 may also be processed by rendering libraries to generate an augmented reality environment that allows the patient to view virtual complications and/or procedure effects on his or her own physical body. The rendering libraries 103 may interface with the GUI 105 to present an augmented reality environment as an interactive model of procedure stages or complications transposed on a live video stream or image of a patient's body. In one example, the patient interacts with the GUI 105 to angle, rotate, or otherwise manipulate the model by moving the area on his or her body receiving the surgical or non-surgical procedure or having the complication. The augmented reality environment provided by the GUI 105 tracks changes in body position and automatically adjusts the 3D model to reflect the changes. Accordingly, the augmented reality environment provides a realistic perspective of post-operative results across a 360° range of rotation and 180° of horizontal tilt.

In other examples, the GUI 105 displays a dynamic 3D model of the patient's body after undergoing one or more stages of a procedure, having a complication after a defined period of time, or after a defined recovery period without complications. In this example, the patient interacts with the GUI 105 by dragging, touching, tapping, or otherwise manipulating the touch screen on the client device 100. These touch screen manipulations move the model in the direction of the manipulation. For example, to rotate the 3D model to the right, a patient may touch the model on screen and drag the model to the right.

The GUI 105 may also include a messaging platform for facilitating communication between two or more client devices 100 running the patient education, consent, and follow-up application. Users may send direct messages including text, images, videos, models rendered in 3D, other any other information relevant to patient education, patient consent, or post-operative treatment. Patients, physicians, insurance companies, and other participants in the healthcare system may all become users of the patient education, consent, and follow-up application by running an instance of the application on a client device 100. Accordingly, patients may use the GUI 105 messaging platform to report compilations, ask questions about educational material, and otherwise communicate directly with their physician. Communication channels between patients and physicians can be private to preserve patient confidentiality. Alternatively, groups of patients may communicate with one or more physicians in a public or semi-private communication channel or forum that allows patients to share information and discuss physician responses collectively within a community of patients interested in the same procedures, located in the same area, having the same physician, or otherwise sharing a common interest.

The GUI 105 may also render an VR/AR environment for visualizing 2D/3D models. The VR/AR environment may include a 2D/3D model editing tool that enables real time editing of 2D/3D models of anatomical features as part of the education process. The model editing tool may be compatible with a touchscreen and/or pen or stylus to enable edits to 2D/3D models by manual drawing of new boundary lines defining the dimensions of one or more anatomical features. The model editing tool may be integrated with a simulation aspect of the VR/AR environment to enable rendering of simulations depicting changes from an original 2D/3D model to an edited 2D/3D model in the GUI 105.

The components included in the server system 120 may be configured to run on one or more servers, virtual machines, or cloud compute instances. The server system 120 components provided functionality to the client devices 120 through an application interface 110 and optionally through one or more integrations libraries 103 which provide and management more complex communications between the server system 120 and the client devices 100, for example, interactive education content and consent workflows from the education and consent application 127, automated diagnoses and diagnostic reports from the artificial intelligence system 125 and interactive follow-up questionnaires and complications monitoring from the complications monitoring service 128.

The sever system 120 components include a communications module 124 that provides a connection to a wireless service 127 as well as a network connection 130 having a security layer for ensuring secure communications between internal or third-party computer systems 132-139 and other Internet applications and the server system 120. The security layer 130 also interfaces with the communications module 124 to authenticate access to a server system network that interfaces with the application interface 110 and client devices 100, The server system further includes a content management system 122 for managing a graphic content library including documents, graphic content, artificial intelligence models, 3D models, simulations, and augmented reality environments and other content produced or processed by the server system 120. The content management system 122 may also selectively provide graphic content, text data, and interactive media to the education and consent application 127 for display in one or more client devices 100 as part of an education course.

The graphic content library and all other platform data is held in a data storage module 121. The data storage module 121 provides physical storage, memory, and backups for the graphic content library and all other platform data generated or processed by one or more server system 120 components. The AR system 125 generates one or more 3D models, simulations, or augmented reality environments from patient measurement data, training data sets, analytics information, digital representations of procedure stages, and virtual complications data provided by the content management system 122. The AR system 125 interfaces with one or more rendering libraries within AR system 125 or alternatively within the client-side integrations libraries to provide 3D models, simulations, and AR environments to the client-side imaging engines 104. Communications between the server system 120 and client-side components required to provide one or more 3D models, simulations, or augmented reality environments to client devices 100 are exchanged over the application interface 110. The server system further includes business logic 123 for performing the day-to-day business tasks of the server and client systems. Tasks performed by the business logic include data analytics, accounting and payment processing, as well as chat and messaging.

One or more internal or third-party services, computer systems, or other applications connected to the Internet may provide data to, or otherwise interface with, at least one of the client device 100 components or the server system 120 components. Example third party services include brand intelligence 132 for providing information about products and product brands used in surgical or non-surgical procedures; business intelligence 133 for providing customer information, customer and physician leads, as well as sales and marketing material and performance; physician intelligence 135 for providing physician analytics including performance history and post-operative results; a measurements service 136 for providing pre-operative and/or post-operative patient measurements as well as procedure action measurements, for example, incision sizes and suture widths, and complications measurements such as infection size and expected growth rate if treated or untreated; patient intelligence 137 for providing patient analytics including demographic information and post-operative results; education materials service 138 for providing educational content relevant to the stages, benefits, risks, and complications of surgical and non-surgical procedures; and complications intelligence 139 for providing complications data including complication images and complication rates associated with a particular procedure, group of patients having particular demographics, physician, practice group, or procedure product brand. Example third party computer systems include a location system 132 that provides location data for patients and physicians, for example, GPS data or street address information, and a patient imaging system for providing pre-operative and post-operative images, video, and other graphic content, and a complications imaging system for providing patient images taken during the recovery process. Data provided by one or more third party services or computer systems may be ingested by one or more of the client device 100 components or server system 120 components to curate and provide educational courses, present and execute patient consent workflows, give automated complications diagnoses, and generate one or more 3D models, simulations, or augmented reality environments. As used herein the term “third party services” may include services or computer systems that are components of the invention described herein, for example, other server-side components running on a virtual machine or cloud-computing instance. As used herein the term “third party computer systems” may include services or computer systems that are components of the invention described herein, for example, other server-side components running on a virtual machine or cloud-computing instance.

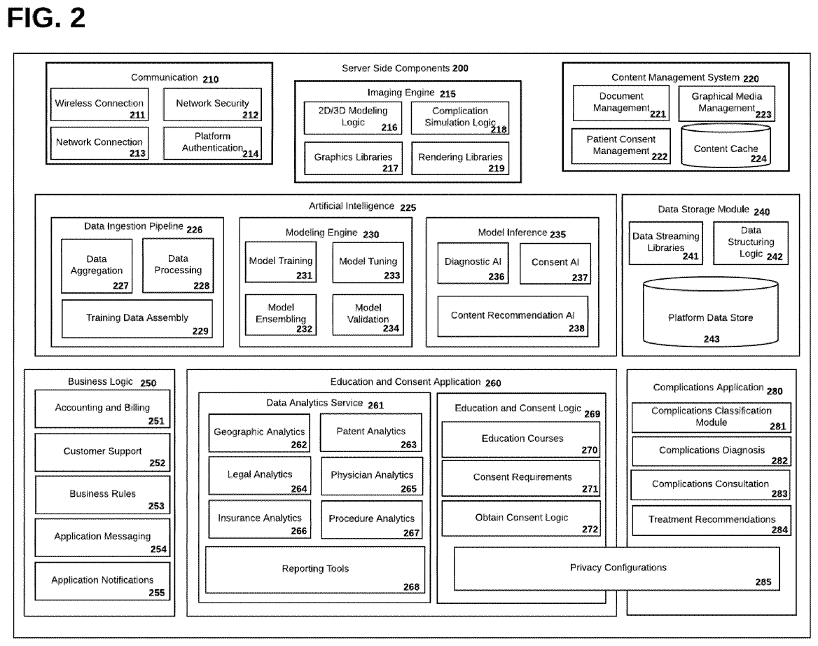

FIG. 2 illustrates a server system for providing a patient education, consent, and post-operative monitoring application. The server system includes a plurality of server-side components 200. A communication module 210 provides a wireless connection 211 and a network connection 213 for interfacing with third party services, computer systems, or other applications connected to the Internet. The communications module 210 also provides security features including network security 212 and platform authentication 214. The communications module 210 interfaces with one or more server-side components to secure platform data and messaging between internal system components, connected devices, third party computer systems, and Internet applications. In one example, the network security module 212 interfaces with an imaging engine 215, Artificial intelligence system 225, business logic 250, education and consent application 260, and patient monitoring application 280, to provide secure data received from one or more third party services, computer systems, or applications connected to the internet. In another example, the platform authentication 214 module interfaces with the artificial intelligence system 225, data storage module 240, and content management system 220 to restrict access to proprietary AI models and confidential patient data.

The server-side components include an imaging engine 215 for generating 3D models, simulations, and augmented reality environments that present realistic representations of surgical and non-surgical procedures and well as associated complications. In one example, 2D/3D modelling logic 216 assembles 3D models of patient bodies after stages of a procedure and at periodic points in the recovery process from textures, graphics, physics constraints, real life images, and virtual representations provided by graphics libraries 217. The graphics libraries 217 may interface with the content management system 220 to retrieve and process graphical content into textures, graphics, and virtual representations that used for making 3D models or simulations. The graphics libraries 217 may also interface with complication simulation logic 218 to provide textures, graphics, physics constraints, real life images, and virtual representations that are assembled into 3D models and simulations of procedure complications. In addition to receiving processed graphical content from the graphics libraries 217, the 2D/3D modelling logic 216 and the complication simulation logic 218 may interface directly with the content management system 220 to retrieve raw graphics content.

The imaging engine 215 also includes one or more rendering libraries 219, for example, 3D model rendering libraries for compiling raw models generated by the 2D/3D modelling logic 216 and the complications simulation logic 218 into cohesive models that are sent to client devices and viewable through a user interface. Additionally, the rendering libraries 219 may AR render libraries for compiling AR environments generated by the imaging engine 215. The rendering libraries 219 further include simulation rendering libraries for compiling several 3D patient models and/or complications models into a cohesive procedure or complications simulation. The simulation rendering libraries may further include simulation streaming libraries for streaming simulations complied from one or more post-operative stage patient models provided by the 2D/3D modelling logic 216 and/or complications models and simulations provided by the complication simulation logic 218. In one example, the streaming libraries provide for simulation streaming over a content streaming network configured for variable or adaptive bitrate streaming. One or more of the 2D/3D modelling logic 216, the rendering libraries 217, or the complication simulation logic 218 may also include matching logic for matching the orientation of the 3D model in an AR environment with the orientation of a patient body part in real time. The matching logic includes one or more libraries for tracking movement of a patient body part in live streaming video and automatically adjusting the 3D model object depicted in the AR environment to dynamically fit the patient's body part. Alternatively, the matching logic may match the orientation of a 2D post-operative stage patient model or a complication model to a 2D picture of digital image or a patient's body. In this example, the imaging engine 215 overlays the virtual model over the 2D photograph or digital image to augment the appearance of the photographed body part with a virtual representation of the desired procedure impacts, complication, or recovery effects.

2D/3D models, simulations, and AR environments generated by the imaging engine 215 are managed by a content management system 220 having a graphical media management module 223. The content management system 220 further includes one or more document management modules 232 for managing documents and other text information processed by the artificial intelligence system 225 or incorporating into education courses by the education and consent application 260. The content management system also includes a patient consent management module 222 for storing and managing patient consent documents presented to- and executed by- patients using the consent aspect of the patient education, consent, and post-operative follow-up application. A content cache stores all documents, text data, graphical media, AI models, 3D models, simulations, AR environments and other frequently used data that must be provided to internal server components or connected devices in less time that it takes to load into memory from cold storage.

The artificial intelligence system 225 provided herein includes a data ingestion pipeline 226, an AI modelling engine 230, and an AI model inference server 235. The data ingestion pipeline 226 includes a data aggregation module 227 that interacts with at least one of the content management system 220, the data storage module 240, or one or more internal or third party data sources to aggregate information about patients, physicians, procedures, and procedure complications in order to diagnose complications and make predictions about likelihood of procedure success and patient recovery time. In one example the data aggregation module ingests patient data including patient personal information, demographics, and physical measurements; procedure data including procedure type, stages within each procedure, risk, success rate, and materials used to perform a procedure; and physician data including physician performance metrics, age, and experience.

The data processing module 228 receives raw data ingested by the data aggregation module 227 and cleans the raw data and formats it for analysis. The data processing module 228 may also classify, tag, sort, and otherwise transform the data for efficient storage, filtering, and streaming. The data processing module may also tokenize data points within a data set into features or map data points to a multi-dimensional space. The training data assembly module 229 generates training data sets for training AI models by selecting a subset of the clean, processed data. Training sets assembled by the training data assembly module may include large datasets containing a massive variety of data points as well as smaller datasets containing more specific data points relevant to the procedure, patient, physician, or situation the AI model is intended to analyse.

The AI modelling engine 230 interfaces with the data ingestion pipeline 226 to retrieve one or more of raw data from the data aggregation module 227, processed data from the data processing module 228, and training datasets from the training data assembly module 229. Data streaming libraries 241 within the data storage module 240 may be used to process, retrieve, or train on very large datasets that cannot be stored entirely or efficiently in system memory. The model training module 231 generates AI models using the training datasets and, in some cases, the raw or processed data provided by the data ingestion pipeline 226. AI models may be generated by the model training module 231 according to one or more machine learning algorithms including data driven natural language processing methods, for example, TF-IDF or bag of words, vector based methods such as node2vec or random walks, image classification techniques such as pixel classification or convolutional neural nets, and deep learning methods, for example, neural networks, hierarchical neural networks, or attention networks. Before being put into production in the AI model inference server 235,

AI models generated by the AI modelling engine 230 must be tuned and validated. The model tuning service 233 manipulates trained models by exposing training parameters and model architecture for modification. Raw and tuned models are tested for accuracy using the model validation service 234 which withholds a portion of the training data and tests model performance based on how well it performs on classifying or predicting results in the withheld training data sample. Tests for accuracy performed by the model validation service to ensure AI models are robust, accurate, and not overfit to the training data before being pushed to a production environment for inference by users and/or other internal systems. AI models generated by the AI modelling engine 230 can be combined with other models using the model ensembling service to generate robust and accurate ensemble models. These ensemble AI models can combine multiple machine learning algorithms and/or artificial intelligence techniques, for example, TF-IDF and neural networks or convolutional neural networks and node2vec models to generate more accurate and robust models. In many cases, ensemble models provide more accurate results than standalone models because of the trade-offs associated with using one machine learning technique over another. Accordingly, combining two or more machine learning techniques that have complementary sets of advantages and disadvantages, for example, training speed and accuracy, or corpus depth and corpus scope, contextual and naïve, or data driven and rules based, can yield a higher performing AI system than using one model or one class of machine learning technique.

The AI model inference server 235 exposes trained AI models provided by the modelling engine 230 for inference by client devices and other internal systems. AI models that are being tested and perfected a served so that they can be tuned and validated while AI models in production are served so that client devices can interact with the AI models to received diagnoses or predications. The artificial intelligence system 225 described herein includes a different AI inference sever for each AI model of class of AI model provided by the server system. In one example, a diagnostic AI server 236 serves AI models that diagnose complications from patient data, for example, symptom descriptions and uploaded patient images. The consent AI 237 predicts the consent requirements that will govern a patient or procedure based on the location of the patient, the location of the physician performing the procedure, and the laws of jurisdiction governing the relevant locations. A content recommendation AI 238 suggests content that should be provided to patients as part of an education course based on patient data, such as, age and demographics, the procedure the patient will undergo, the physician performing the procedure, the materials used in the procedure, and the consent requirements of the relevant jurisdiction. These are just three examples of AI models generated by the AI modelling engine 230 and served for inference by the AI model inference server 235.

Many other models within the scope of this invention can be created from the data ingested by the data ingestion pipeline 226. In one example these AI models predict procedure success rates and physical changes that will occur as a result of a procedure bases on automated analysis of patient measurements, historical post-operative results data, procedure information, and historical physician performance. One or more artificial intelligence models or machine learning algorithms provided by the artificial intelligence system 225 may interface with the AR system to generate more accurate 3D models, the simulations, and AR environments that provide patients with a more realistic understanding of the physical impacts of undergoing procedures and the effects of post-operative complications.

The education and consent application 260 curates education materials and relevant legal regulations to provide education courses and patient consent workflows to the software application. The education and consent application 260 includes a data analytics service 261 for sorting and selecting information and education and consent logic 269 for assembling information provided by the data analytics service 261 into education courses and patient consent workflows. The data analytics service 261 may interface with the content management system 220 and/or data storage module 240 to provide instructions for providing content to the education and consent logic 269 according to the results of analysis performed by one or more modules within the data analytics service 261. Additionally, results from analysis performed by the data analytics service 261 and education courses and patient consent workflows assembled by the education and consent logic 269 may be provided to the data storage module 240 and/or content management system 220 for storage and distribution to client devices.

The data analytics service 261 interfaces with the data ingestion pipeline 226 to collect and process data from one or more internal or third-party data sources. Specialized modules within the data analytics service 261 then analyse data received from the data ingestion pipeline 226 by sorting, grouping, counting, tagging, graphing, filtering, and otherwise transforming the data. Modules within the data analytics service 261 may be configured to transform a particular data type, for example, a geographic analytics module 262 for performing analysis on location information received from patient and physician devices to determine the physical location of patients and physician offices; a legal analytics module 264 for performing analysis on legal data including patient consent regulations and other medical compliance data to determine what patient consent regulations apply to a particular patient based on the type of procedure they are undergoing and the jurisdiction governing the procedure; and an insurance analytics module 266 for analysing patient and healthcare provider insurance information to determine the consent requirements for medical insurance reimbursement for a particular patient, geographic location, and/or insurance provider. Other specialized analytics modules within the data analytics service 261 may include a patient analytics module 263 for performing analysis on patient data to classify patients into groups based on one or more parameters, for example, demographics, geographic location of residence, procedure type, or medical history; a physician analytics module 265 for performing analysis on physician data to classify physicians into groups based on one or more parameters, for example, demographics, geographic location of practice, areas of expertise, performance history, procedure complications record or years of experience; and a procedure analytics module 267 for selecting education content and consent workflows required to enhance patient understand the risks and complications associated with a particular procedure and satisfy the informed consent requirements for a particular procedure.

Results generated by the data analytics service 261 may be provided to other internal or third-party systems in raw data form delivered via API calls or some other scripted distribution method. Alternatively, data analytics generated by the data analytics service 261 may be complied by one or more reporting tools 268 and delivered as a report document, for example, a Word or PDF document. Reports provided by the reporting tools 268 may contain graphs, charts, tables and other visualizations as well as text analysis. Reports may also be included in education courses generated by the education and consent logic 269.

The education and consent logic 269 includes program instructions for assembling education courses 270 and consent workflows 271 from analytics data provided by the data analytics service 261. Education courses 270 generated according to instructions provided by the education and consent logic 269 may include, for example, images, videos, 3D models, 2D models, simulations, and other graphical media content as well as text descriptions, analysis, audio recordings, and other non-graphical media. Graphical media content and non-graphical media content contained in the education courses 270 may be obtained from internal sources such as the content management system 225 and/or the data storage module 240 as well as third party computer systems and Internet applications. Content included in the education courses 270 may be informed by data analytics results to be specific to a particular procedure, patient, group of patients, or physician. Analytics results informing the selection of education content by include insights into what content is most likely to engage, inform, or reduce complications for a specific patient or group of patients. Education courses 270 may be assembled by the education and consent logic 269 according to one or more manually defined or machine learned criteria provided by the artificial intelligence system 225 and/or the data analytics service 261, for example, procedure, patient characteristics, patient post-operative results, patient pre-operative measurements, physician post-operative results, patient demographics, patent location, physician location, physician post-operative results relative to other physicians and/or practice groups, physician practice group, practice group size, and/or practice group post-operative results.

Consent workflows 271 generated by the education and consent application 260 may include a selection of jurisdiction specific disclosures or manifestations of consent as required by the regulatory regime governing a specific procedure. The required disclosures and allowable manifestations of consent included in the consent workflows 271 by be programmatically determined based on instructions contained in the informed consent logic 272. The informed consent logic 272 may interface with the geographic analytics module 262 and legal analytics module 264 to determine the legal jurisdiction governing a particular procedure and the patient consent requirements within the applicable legal jurisdiction. This legal jurisdiction information and applicable consent requirements are then incorporated into a consent workflow 271 by the informed consent logic 272.

Example consent workflows 271 may include a questionnaire having text or recorded descriptions of procedure risks and complications as well as the physical impacts and body trauma that may occur during the procedure. Procedure risks, complications, physical impacts, and body trauma may also be presented as a 2D/3D model or simulation showing the complication, physical impact, or trauma on a digital image of the patient's actual body. The consent workflow 271 may include an interactive response for the patient to manifest their approval or disproval of each questionnaire prompt such as a check box, clickable button, or free form text input box. Additionally, the consent workflow 271 may enable patients to download a consent form, sign the form offline in ink, and upload a signed form to the consent application. Other means of manifesting consent may also be provided to the patient by the education and consent logic 269 including an option to digitally sign a consent for and/or record affirmative responses to questionnaire prompts and a statement that the patient agrees on all terms, and that he or she understands the procedure and its potential risks and complications.

Signed consent forms, consent recordings, and questionnaire responses obtained from patients may be stored in the data storage module 240 for future reference. Additionally, the consent materials may be shared with the submitting patient, insurance companies, clinics, hospitals, or other physicians according to privacy configurations 285. In one example, the privacy configurations 285 are determined by the patient and apply to all patient data on the platform. In other examples, the privacy configurations 285 are determined by the healthcare provider or health insurance company and must be shown to- and agreed to by the patient before the patient can use the education, consent, and monitoring application.

To control use and sharing preferences of all patient data on the education, consent, and monitoring application, the privacy configurations 285 must interface with the patient monitoring application 280. The patient monitoring application 280 collections post-operative patient information to monitor recovery and diagnose complications. A complications classification module 281 interfaces with one or more AI models generated by the artificial intelligence application 225 and/or data analytics modules provided by the data analytics service 261 to determine the complications most likely to impact the patient based on the procedure type, patient characteristics, and physician complication rate. Based on this analysis the complications consultation module 283 generates a custom recovery consultation questionnaire for the patient to fill out periodically during their recovery period. In one example, to complete this questionnaire the patient must upload one or more pictures of the body part or parts impacted by the procedure. The pictures as sent to the complications diagnosis module 282 for an automated diagnosis. To make the diagnosis the complication diagnosis module 282 may inference the Diagnostic AI models 236 served by the AI model inference server 235.

Based on the results of the automated diagnosis the treatment recommendation module 284 suggests a recommended treatment plan to remedy a complication or continue with recovery. For example, patients having complications diagnosed by the complications diagnosis module 282 may be scheduled for an office visit with a physician by the treatment recommendation module 284. Alternatively, the treatment recommendation module may suggest an at-home remedy, for example, cleaning a procedure site, restricting a certain type of activity, or treating the procedure site with an over the counter medicinal product. In situations where the complications diagnosis module 282 is unable to diagnose a complication or determine with a high degree of certainty that no complication exists the treatment recommendation module 284 may escalate the patient's case to physician for a human review of the images submitted by the patient. When no complication is diagnosed at a high degree of certainty, the complication diagnosis module 283 provides peace of mind the patient be assuring him or her that recovery is going well.

The server system 200 further includes business logic 250 for performing the day-to-day business tasks of the server and client systems. Accounting and billing libraries 251 interface with client devices to process payments, generate pay history, and track invoices. Customer support libraries 252 interface with the client devices to provide customer service and troubleshooting. Business rules 253 provide frameworks and protocols for managing the day-to-day operations of the client and server systems. In one example, business rules include a patient profile management system that interfaces with the patient database stored on the platform data store 243 to efficiently provide patient data to the analytics service 261. The business rules 253 also provide a physician profile management system that interfaces with the physician database stored on the platform data store 243 to efficiently provide physician data to the analytics service 261. The business logic 250 also includes components for sending messages and interacting with third party services, computer systems, or applications connected to the Internet. More specifically, the application messaging service 254 provides email and chat to the client application. The application notification service 255 provides push notifications to the client application to alert users to events that occur within the client application, for example, receiving a message or obtaining access to a new 3D model, simulation, or AR environment. The other components of the business logic 250 may include an integrations service that interfaces with the communications module 210 to provide integration configurations for third party services, computer systems, and other applications connected to the Internet that interface with client and server systems. The integration configurations improve the interoperability of the client and server systems with third party services, computer systems, and other applications connected to the Internet that interface with client and server systems, for example, a social media application or payment platform.

The server system 200 also includes a data storage module 240 having a platform data store 243 that provides storage, memory, and backups for all platform data. The analytics service provides quantitative metrics, visualizations, graphs, and other analytics content to the imaging engines 215 and client application. The data storage module 240 may also include data streaming libraries 241 for providing large data sets to one or more internal components or third-party computer systems as well as data structuring logic 242 for efficiently storing a high volume of data across many different data types.

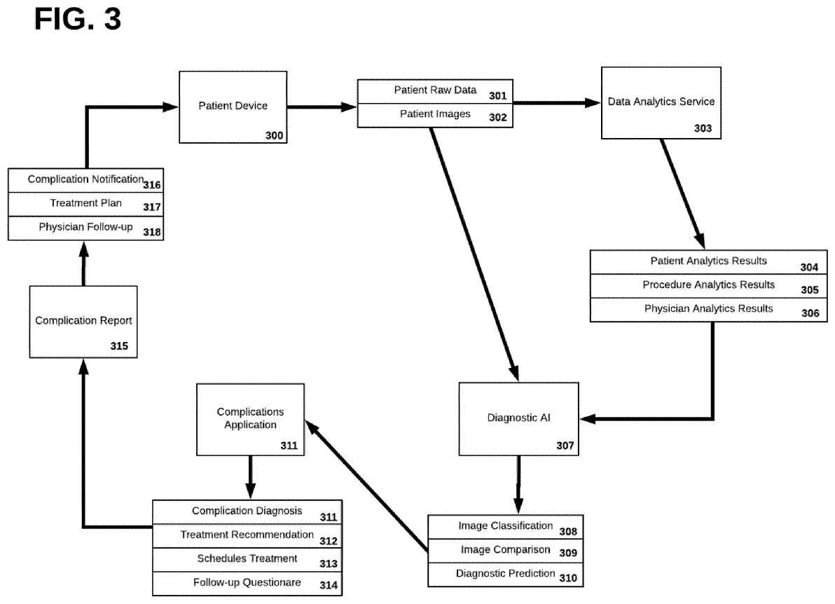

FIG. 3 illustrates an example process for making automated complications diagnoses on post-operative patients. Patients use a patient device 300 running an instance of the patient education, consent, and follow-up application to answer a post-operative questionnaire, describe their symptoms and take one or more photographs of their body showing the areas impacted by the procedure. The patient raw data 301 and patient images 302 received from the patient are then uploaded to the data analytics service 303 and diagnostic AI 307 components of the server system. The data analytics service processes the patient raw data 301 to generate patient analytics results 304, procedure analytics results 305, and physician analytics results 306 that are used to better inform the inference made by the diagnostic AI 307 as well as enhance the patient's recovery profile and update the doctor and procedure statistics to improve the data analytics process performed by the data analytics service 303. The updated analytics results may also be incorporated into a training dataset that is used by the artificial intelligence system to train an updated version of one or more AI models, for example, the diagnostic AI model 307.

To diagnose a complication using the uploaded patient images 302 and raw and processed patient data, the diagnostic AI 307 classifies the image as containing a complication or not using one or more AI models, for example, a convolutional neural network containing one or more layers of machined learned image classification parameters. The diagnostic AI 307 may also compare patient images 302 to one or more repositories of images tagged as containing complications and images tagged as not containing complications to diagnose a complication. The image classification model 308 and image comparison model 309 may also be combined through a weighted or unweighted ensembling process to leverage the predictive power of each model in making a diagnostic prediction 310. Diagnostic predictions made from inferencing the diagnostic AI 307 are sent to the complications application 311 to assemble a complications report 316.

In one example, the complications report 316 contains a complication diagnosis 312 made using the diagnostic prediction received from the diagnostic AI 307 and data analytics results, for example, the statistical probability of the predicted complication for a specific patient, physician, procedure, or brand of procedure product. The complications application 311 may further generate a treatment recommendation 313 based on the statistical probability of cure by a given treatment for a specific complication, patient, physician, procedure, or brand of procedure product. For treatments that need a consult or prescription with a physician, physical therapist, or other healthcare provider, the complication application 311 may schedule a treatment appointment 314 based on the mutual availability of the patient and the healthcare provider. The selected appointment time or alternatively, for home treatments, a home treatment schedule containing the days and time to administer home therapies may be included in the complication report 316. Additionally, based on the complication diagnosis 312, treatment recommendation 313, and treatment schedule 314, the complications application 311 will generate a follow-up questionnaire 315 designed to monitor the patient's specific recovery process and any complications treatment in situations where a complication was diagnosed. The follow-up questionnaire 315 may include, for example, appointment reminders, check lists for administering complications treatments, descriptions of related complications to watch out for, descriptions of more serious complications that a patient with a less serious complication has a greater risk of experiencing, contact information of healthcare providers, and reminders for completing the next periodic post-operative questionnaire on the patient device. Follow-up questionnaires 315 may be customized depending on previous surveys' answers. Additionally, depending on the answers provided by the patient on the follow-up questionnaires 315, communication between patient and physician may be automatically to speed complication diagnosis and make treatment more efficient.

The complications application 311 packages the complications diagnosis 312, the treatment recommendation 313, treatment schedule 314, and follow-up questionnaire 315 in a complications report 316 delivered to the patient device 300 via email message, push notification, or internal message within an instance of the education, consent, and monitoring application running on the patient device 300. In situations where the compilations diagnosis 312 includes a diagnosed complication, delivery of the treatment report may be concurrent with other communications, for example, a complication push notification 317 containing a complication alert, a complication treatment plan 318, and a physician follow-up appointment 319 or a text or recorded description of advice for treating the complication from a physician.

In some embodiments, the diagnostic AI 307 may ingest patient raw data 301 and patient images 302 to generate a prediction about the appropriate timing for future procedures. Specifically, the diagnostic AI 307 may indicate the appropriate time for administering fillers and/or botox or other cosmetics or performing other procedures that occur on a reoccurring basis. To generate a prediction indicating the time to perform a procedure, the diagnostic AI 307 synthesizes patient images 302 uploaded to the patient education, consent, and follow-up application over time. By comparing a feature map including one or more points and/or vectors describing critical areas of interest in an image isolated from the most recent patient images with the corresponding feature map extracted from earlier uploaded patient images, the diagnostic AI 307 tracks the change in a patient's appearance over time. The diagnostic AI 307 may suggest a time to perform a reoccurring procedure proximate to the time wherein the feature map generated by the diagnostic AI 307 from a newly uploaded patient image falls outside a range of similarity with a feature map generated from earlier uploaded patient images. In some embodiments, the range of similarity is a customizable setting variable by users of the patient education, consent, and follow-up application.

Initially, the diagnostic AI 307 is trained on a library of images collected from a wide range of people having a comprehensive range of body types, facial features, and skin tones. The initial training dataset for the diagnostic AI 307 may also include synthetic images comprising computer generated faces produced using facial features isolated from real face photos of people. Over time, as the patient uploads more images, the model will be retrained on their own data and predictions will be more accurate. Images uploaded to the patient education, consent, and follow-up application may be shared with the patient's doctor or other provider. Patient images 302 may be synthesized manually by the provider or synthesized using a machine learning/manual hybrid approach to determine when the patient should get a procedure or receive treatment for a complication. Manual synthesis of patient images 302 may be frequently used to determine when cosmetic products including fillers and/or botox are absorbed and should be re-applied. Medical advice including a provider's diagnosis and/or guidance may be shared through the patient education, consent, and follow-up application.

In some embodiments, the database of patient images 302 and videos may be specific to particular procedures and products used in procedures. The database of patient images 302 may be associated with timestamp information describing a date and time a procedure was performed and a time period between administering a particular product. The augmented database of patient images 302 and raw patient data 301 may be used to train the diagnostic AI 307 to perform a variety of tasks. In one embodiment, patient images 302 and raw patient data 301 ingested by the diagnostic AI 307 may include images of partners, children and other relatives. By synthesizing information of relatives, the diagnostic AI 307 may perform a genealogical analysis to predict the effects of performing particular cosmetic procedures as well as the likelihood of contracting infections and diseases.

The diagnostic AI 307 may generate an anatomical morphology model improving accuracy of visual renderings simulating after effects of one or more cosmetic procedures on facial features and body parts. In other examples, the diagnostic AI 307 may generate a manufacturing model providing feedback to manufacturers about the durability, effects, and patient satisfaction of their cosmetic products. The diagnostic AI 307 may also generate a model providing feedback to healthcare providers including patient conversion rates, patient satisfaction rates, and product information. In other examples, the diagnostic AI 307 may use the library of patient images 302 and demographic information to generate models predicting occurrences of procedure compilations, infections by pathogens, and serious diseases based on patient skin characteristics and body shapes. The diagnostic AI 307 may also generate aging models predicting the occurrence of aging characteristics in a patient based on their geographic location and any previously performed cosmetic procedures.

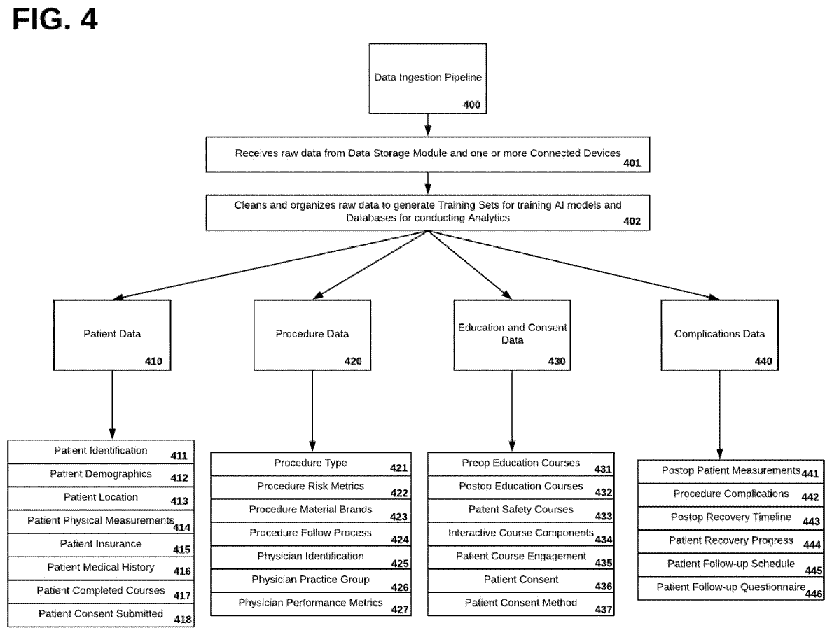

FIG. 4 illustrates a data map containing various data types processed by the patient education, consent, and follow-up application. Data processed by the server system of the application is aggregated using a data ingestion pipeline 400 that receives raw data from one or more internal system components, third party computer systems, Internet applications, or connected client devices 401. Optionally, the data ingestion pipeline cleans and organizes raw data to generate training sets for training AI models and databases for conducting data analytics 402.

Data types ingested by the data ingestion pipeline 400 include patient data 410, for example, patient identification information 411, patient demographics information 412, patient location information 413, patient physical measurements 414, patient insurance information 415, patient medical history information 416, patient completed courses 417, and submitted patient consent documents. Procedure data 420, for example, procedure type 421, risk metrics 422, material brand 423, recovery metrics such as recovery timelines, recovery rates, and complications rates, identification information for the physician performing the procedure, the practice group of the physician performing the procedure, and the performance metrics of the physician performing the procedure, is another data type ingested by the system. The data ingestion pipeline 400 also processes education and consent data 430 including pre-operative education courses 431, post-operative education courses 432, patient safety courses 433, interactive course component types and interaction rates 434, patient course engagement metrics 435, patient consent documents 436, and patient consent methods 437. The data ingestion pipeline also processes complications data 440, for example, post-operative patient measurements 441, procedure complications metrics 442, procedure recovery timelines 443, patient recovery progress 444, patient follow-up schedule 445, and patient follow-up questionnaires 446.

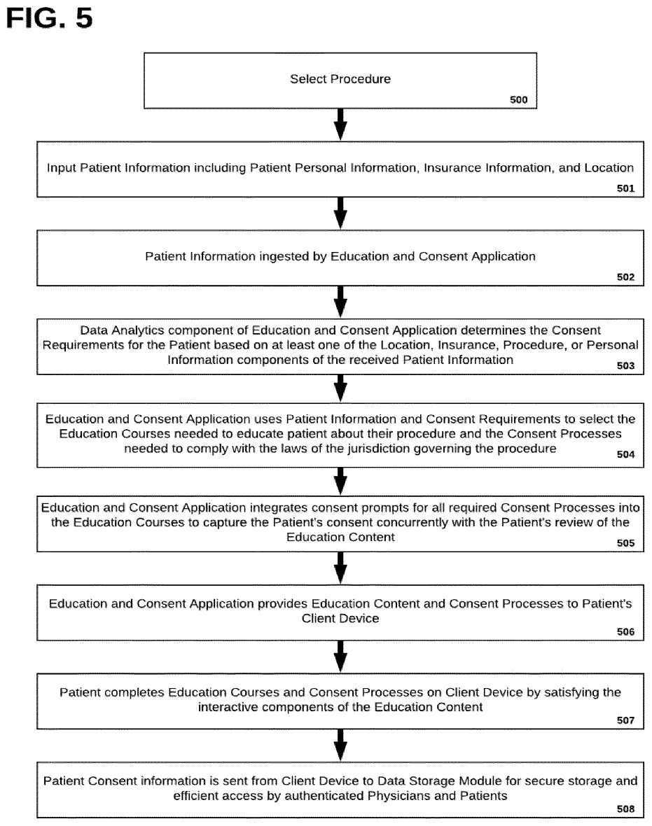

FIG. 5 illustrates an example consent workflow generated by the computer system described herein for obtaining informed patient consent. To begin the workflow, a patient selects a procedure to consent to 500. Alternatively, the procedure information may be preloaded into the consent workflow based on information received from previous patient consultations. If not also preloaded into the system, the patient then inputs patient information included patient personal information, insurance information, and location information 501 into a patient device running an instance of the patient education, consent, and follow-up application. The input patient information is then ingested by the education and consent application 502 within the server system. Specifically, the data analytics component of the education and consent application determines the consent requirements of the patient based on at least one of the location, insurance, procedure, or personal information components of the received patient information.

The education and consent application then uses patient information and the determined consent requirements to select the education courses needed to educate the patient about her procedure and the consent processes needed to comply with the laws of the jurisdiction governing the procedure 504. Next, the education and consent application integrates consent prompts for all required consent processes into the education courses to capture the patient's consent concurrently with the patient's review of the education content 505. The education and consent application then provides the education content and consent processes to the patient's device 506. Once the patient receives the education content and consent workflow, on her device, the patient completes the education courses and consent processes on her device by satisfying the interactive components of the education content 507. Patient consent information is then sent from the patient device to the data storage module where the consent information is securely stored and accessible by authenticated physicians and patients 508.

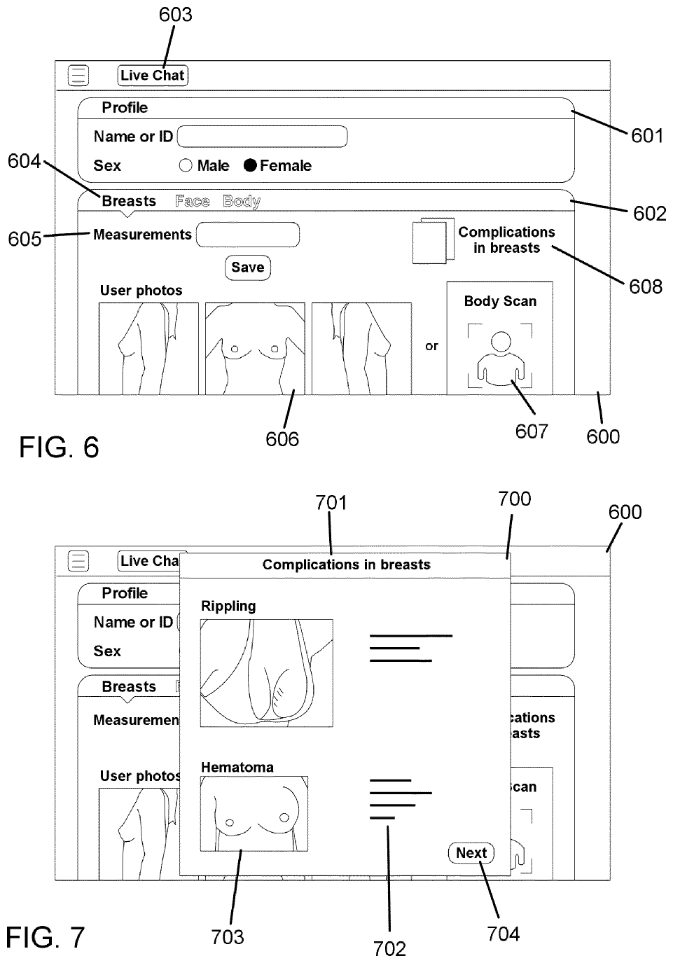

FIGS. 6-9 illustrate one example user interface implementation of the e-learning platform and patient consent and follow-up application. FIG. 6 shows an example home page 600 having a patient identification or profile section 601 in the upper portion of the page and a procedure identification section 602 in the lower part of the page. A chat button 603 is also included. Clicking the chat button 603 launches a live chat application or opens up a messaging modal for communicating with an expert about a procedure. The patient identification section 601 includes, for example, a free form text box for entering a patient name or ID number and clickable radio button for selecting the gender of the patient.

The procedure identification section 602 includes one or more procedure selection tabs 604 for selecting a procedure to view in the e-learning platform. To simulate a procedure using the e-learning platform, users enter requested measurements using one or more measurement input boxes 605 and upload images to the platform via the image selection bar 606. Successfully uploaded images appear in user image panels in the image selection bar 606. Clicking the body scan icon 607 will alternatively launch a body scan application in a connected device to collect 3D body scans of a patient's body to use as image data.

Once sufficient measurement data and image data has been provided, a procedure may be simulated using the AR system contained in the application. In one example, the simulation will appear in the user image panels 606. In another example the simulation will appear in a separate screen or within a pop out modal on the home page 600. Procedure simulations may be saved for record keeping purposes or shared by the patient to one or more social networks. The simulations may be implemented in an AR environment that displays accessories over the patient's image such as glasses, clothes, etc, in addition to the procedure simulation. Complications associated with a procedure may also be simulated in a 3D simulation or AR environment. The AR environment may also be part of a community of users, where patients or physicians could view in AR the procedure results or complications observed by other members of the community. In addition to the system described herein the AR environment could be also implemented through hologrammatic systems

The procedure identification section 602 also includes an education course button 608 for launching an education course corresponding to the procedure selected by the user in the procedure selection tab 603. FIG. 7 displays an example home screen 600 having an e-learning platform education course pop out modal 700. The modal includes the title of the education course 701 in the header of the modal. Text descriptions 702 and images 703 are provided in the main portion of the modal. In this example, the education course contains information on complications with breast augmentation procedures such as rippling or hematomas. Clicking the next button 704 in the lower right portion of the modal will display the next slide of the education course in the modal.

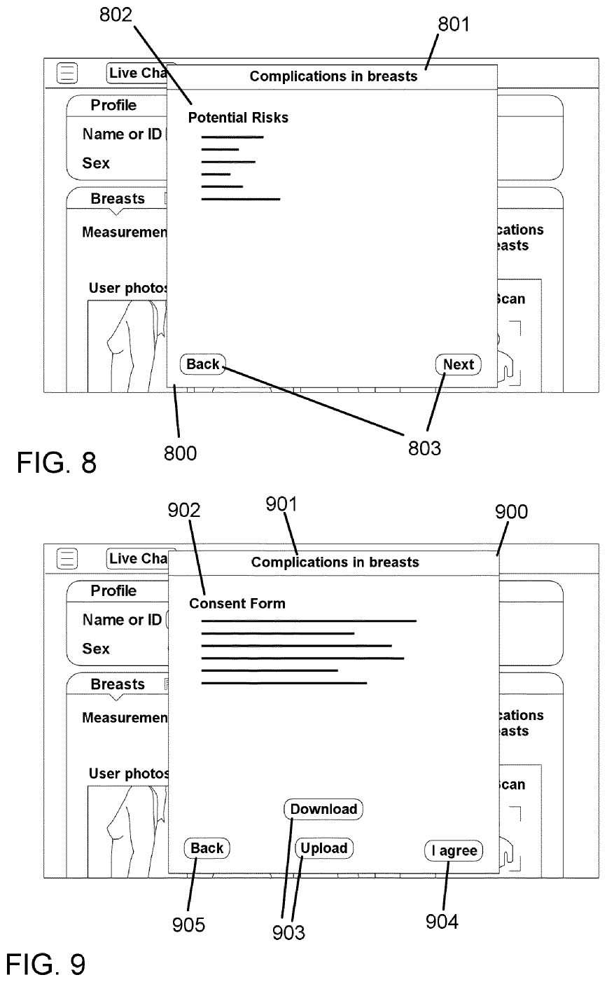

FIG. 8 depicts an example subsequent slide 800 in the breast augmentation compilation education course. The course title 801 is included in the modal header with the education material just below in the main body of the modal. The education material shown in this example slide is a text description 802 of the potential risks and complications associated with breast augmentation procedures. In this example, the text description 802 includes a bullet point list of complications and risks beneath a section heading. The lower portion of the modal includes back and next buttons 803 for accessing the previous slide and following slide in the education course, respectively.

FIG. 9 depicts an example patient consent modal 900 that is made accessible to patients by the e-learning platform and patient consent and follow-up application. The consent modal includes the course title 901 in the header of the modal. The main body of the modal contains a consent form 902 including, for example, links to further education materials and courses, a statement of consent, the name and address of the physician and practice group receiving consent from the patient, and any other relevant information. Clicking the links will display the further education materials in the e-learning platform. The statement of consent requests confirmation that the patient understands the procedure and any associated risks and complications and gives consent to undergo the procedure. Below the consent form are download and upload buttons 903 for downloading a copy of the consent agreement and uploading a signed copy of the consent agreement. An agree button 904 allows the patient to digitally consent to the procedure. In addition to physical and digital signatures, patients may manifest consent by recording a video of the patient reciting a consent statement. A back button 905 exits the consent modal and returns to the previous education course modal.

The e-learning platform and patient consent and follow-up application described herein can be used remotely by the patient or during a consultation with the physician. Optionally, the system may be customized by the physician or labelled with the physician's practice group or brand. The system may also be customized by country or region to account for different legislations and specificities. The system can include representation of physical impacts of a procedure and potential complications and risks directly on a 3D simulation of the patient to enhance their understanding. The e-learning platform can also be linked to medical research institutions or third party societies or companies that provide procedure education materials and courses.

Aspects of the invention described herein can be applied to gain insight into surgical and non-surgical procedures for cosmetic and/or reconstructive purposes. Some example non-traditional surgical and non-surgical procedures that can be simulated by the invention described herein include iris colour implants, hair replacement, weight-loss and or bariatric surgery, fitness, orthodontics and other dental procedures.

Regarding iris colour implants that involve physically changing the colour of the iris. The e-learning platform can provide education courses that make patients fully aware of all colour options available so he can confidently decide which colour to choose. Additionally, the e-learning platform can simulate any potential complication associated with this delicate procedure, so the patient fully understands the risks associated with it.

Regarding hair replacement, this e-learning platform and consent and follow-up application includes the option to generate a 3D model of the patient's head using a number of photos or a 3D scanning device, then provide with the required simulation and planning tools to perform hair transplantation. The system auto-detects the areas from the 3D model with hair and without. The system provides area measurements from the 3D model, either automatically or following manual selection. The system includes different parameters that can be selected by the physician to more accurately simulate the procedure or complications, for example, the density of hair per square centimetre, type of hair, colour of hair, etc. The system also determines the total amount of hair to be transplanted depending on the area and parameters selected. The system provides a 3D representation of the possible results after the transplantation and complications that could occur as a result of the procedure. Both results can complications simulations can be generated to show the evolution of the results or complications over time from first day after procedure or final result. The system provides a catalogue of hair-cuts, styles, colours, etc. for the patient to get more realistic understanding of what he will look like after the procedure.

Regarding weight-loss and bariatric surgery, the e-learning platform and consent and follow-up application includes an option to generate 3D simulations of the patient's body during a weight-loss or bariatric procedure. Complications that may occur as a result of the procedure may also be simulated. The system provides anatomical measurements and volumetric information in an automatic or manual way, to indicate which areas to measure to generate the simulation. The system simulates the evolution of the body during the procedure or after experiencing a complication over time based on the bariatric procedure selected or based or weight-loss plan. The system generates a 3D model of the body at different time steps allowing patients to compare different weight loss procedures and the lasting effects of any complications associated with a particular procedure. Simulations provided by the system are customizable depending on the patient's anatomy, physiology, and physician, procedure, and procedure product material parameters.

Regarding fitness, the e-learning platform and patient consent and follow-up application includes the option to generate the 3D simulation of the patient's body over time after the effects of adopting a physical fitness plan. The system provides anatomical measurements and volumetric information in an automatic or manual way and indicates which body areas the user must measure in order to generate a simulation. The system simulates the evolution of the body over time, with specific physical changes incorporated into specific muscle groups trained as part of the fitness plan. The system includes a configurable catalogue of customizable fitness plans and training regimens. Optionally, the system can analyse a body shape and automatically propose a training plan to achieve desired results. The system generates 3D model of body parts at different time steps to compare intermediate stages of fitness with the final product. The system provides a patient specific model that may be customized according to the patient's anatomy and physiology.

Regarding orthodontics and other dental procedures, the e-learning platform and patient consent and follow-up application can generate 3D simulations of orthodontic and other dental procedures as well as any associated complications. The system can be combined with other imaging techniques such as combi CT, MRI, etc. by fusing the results of the imaging or scan into the 3D simulation to provide more realistic simulations to the user. The platform includes a catalogue of different types of teeth, with different shapes and colours, which can be used to simulate the look of different teeth on the face of the patient. The system may generate simulations based on moulds or modals to teeth uploaded by the user. Simulations may incorporate dynamics facial movements such as talking, smiling, or chewing allowing the user to determine how her face will look after the procedure in a variety of circumstances. The system provides measurements of distances, angles, volumes and proportions automatically to inform physicians. The system includes the option to simulate the oral procedures, for example, braces or other realignment procedures, and any potential complications over time allowing the user to compare the shape or her teeth and face before the procedure, at intermediate steps, and after the procedure. The system includes the option to show the simulation over the video stream of the patient in real time using Augmented Reality techniques.

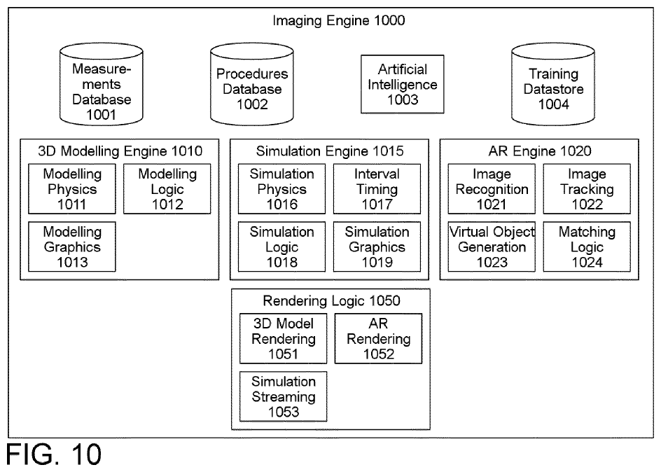

FIG. 10 illustrates an example imaging engine 1000 in more detail than the illustration provided in FIG. 2. The imaging engine 1000 generates 2D images, 3D models, simulations, and augmented reality environments. The imaging engine 1000 includes one or more artificial intelligence libraries 1003 that interact with at least one of a measurements database 1001, procedures database 1002, or a training datastore 1004. In one example, the artificial intelligence libraries 1003 ingest patient measurements and post-operative results data from a measurements database 1001 as well as procedure information and physician results from a procedures database 1002 to generate one or more training data sets held in the training datastore 1004. Artificial intelligence models or machine learning algorithms provided by the artificial intelligence libraries 1003 interface with the 3D modelling engine 1010 to generate 3D models, with the simulation engine 1015 to generate simulations, and with the AR engine 1020 to generate AR environments. The 3D modelling engine 1010 includes modelling logic 1012 that interfaces with one or more artificial intelligence libraries 1003 to generate 3D models using graphics data from the modelling graphics libraries 1013 and physics data from the modelling physics libraries 1011. The simulation engine 1015 includes simulation logic 1018 that interfaces with one or more artificial intelligence libraries 1003 to generate simulations using graphics data from the simulation graphics libraries 1019, physics data from the simulation physics libraries 1016, and timing data from the interval timing libraries 1017. The AR engine 1020 includes image recognition 1021 and image tracking libraries 1022 that identify and track patient body parts included in live streamed video or image content received from a client application running on a client device. The AR engine 1020 further includes virtual object generation libraries 1023 for generating a virtual 3D model object within an augmented realty environment as well as matching logic 1024 for matching the virtual 3D object to a patient body part included in a live streamed video or image content received from a client application. The matching logic 1024 further includes program instructions for tracking movement of a patient body part in live streaming video and for automatically orienting and adjusting the virtual 3D object in the AR environment to dynamically fit the patient's body part.

The imaging engine 1000 further includes or otherwise interfaces with rendering logic 1050. For example, the rendering logic 1050 may be one of the server-side components 200, or the final rendering may be performed client-side. The rendering logic 1050 includes instructions for interfacing with one or more server-side components 200 to generate an AR rendering of a surgical or non-surgical procedure and display the AR rendering on a display device (e.g., an augmented reality display such as a head mounted display (HMD) or AR glasses device, or on a display of a smart phone, tablet or other computing device). The rendering logic 1050 includes 3D model rendering libraries 1051 for compiling 3D models generated by the 3D modelling engine 1010 and AR rendering libraries 1052 for compiling AR environments generated by the AR engine 1020. The rendering logic 1050 further includes simulation streaming libraries 1053 for streaming simulations provided by the simulation engine 1015 over a content streaming network.

In one embodiment, a processor (e.g., a specialized graphics processor) in the server computer system receives procedure information for a surgical or non-surgical procedure selected from the procedures database 1002, patient identifying information, patient measurements selected from a measurements database 1001. The 3D modelling engine 1010 then ingests the procedure information, patient measurement data, patient identifying information, and 3D modelling parameters. Using the ingested data, the 3D modelling engine 1010 then generates a 3D model.

In one embodiment, the 3D model comprises a three-dimensional mesh structure covered in a texture material. The three-dimensional mesh structure may include a patient body part affected by the surgical or non-surgical procedure identified in the procedure information. In one embodiment, the three-dimensional mesh structure may include a collection of points and/or polygons having the same shape and dimensions as the patient body part affected by the surgical or non-surgical procedure. In one example the 3D modelling engine 1010 generates a three-dimensional mesh structure of the patient body part according to body part dimensions provided by the patient in patient measurement data stored the measurements database 1001. In other examples, a three-dimensional mesh structure of the patient body part is extracted by the 3D modelling engine 1010 from a 3D scan of the patient body part generated using a 3D scanning application running on a 3D scanning device. In these examples, the dimensions of the 3D scan may be equal to the dimensions of the patient body part. The 3D modelling engine 1010 may also convert dimensions of the 3D scan to dimensions of the patient body part by performing a conversion operation using a scale factor defined by the 3D scanning application.

The three-dimensional mesh of the patient body party may be covered by a texture material extracted as a texture file by the 3D modelling engine 1010 from a patient photo capturing the patient body part. In other examples, the 3D modelling engine 1010 may select extract the texture material as a texture file from the 3D scan of the patient body part. To ensure the desired area of the texture material cover the correct portion of the three-dimensional mesh structure of the patient body part, the 3D model may include five or more attachment points for fixing the texture material wherein the attachment points map to an area of the texture material included in a texture file. 3D model parameters used by the 3D modelling engine 1010 to construct 3D models of patient body parts include the number of points and/or polygons in the three-dimensional mesh structure, the resolution of the texture material, and the number of attachment points for the textural material.

In other embodiments, the 3D modelling engine 1010 may generate a series of 3D models comprising a pre-operative 3D model illustrating the patient body part before the surgical or non-surgical procedure; a post-operative 3D model illustrating the patient body part after successful performance of the surgical or non-surgical procedure and patient full recovery; and an operative transition 3D model illustrating the patient body part with a partial effect of a successful performance of the surgical or non-surgical procedure. The texture materials applied to each model in the series of 3D models may be the original texture material extracted from the patient photo and/or 3D scan. In some embodiments, the texture material may be expanded or contracted to fit the post-operative and operative transition 3D models. In some embodiments, the 3D modelling engine 1010 may generate multiple transition 3D models, wherein each transition 3D model illustrates a different transition phase of the surgical or non-surgical procedure.

To generate a procedure simulation, the simulation engine 1015 appends animations to the 3D model to provide a simulation of the surgical or non-surgical procedure. In one embodiment, animations modify the 3D model according to anticipated results of the surgical or non-surgical procedure as defined by a practitioner in the procedure information. One example animation may include an expansion or reduction in the size of the patient body part shown according to pre-procedure and post-procedure body part measurements provided by a practitioner. Another example animation may include making an incision into a patient body part and inserting a product into the incision to have the desired effect. A third example animation includes a 3D procedure simulation illustrating a transformation of the patient body part as a result of successfully performing the surgical or non-surgical procedure.

To generate the complete the AR rendering, the AR engine 1020 may transform the simulation of the surgical or non-surgical procedure into an AR rendering of the surgical or non-surgical procedure by mapping points included in the 3D model with corresponding points on the patient body part. The AR rendering may be displayed on an AR display (e.g., an HMD) and/or a mobile electronics device. In other embodiments, the AR rendering may be displayed on a virtual reality display configured to display multiple perspectives of the AR rendering though an intuitive process. In some embodiments, the AR engine may sync the animations included in the procedure simulation with movements of the patient body part. In other examples, the AR engine 1020 may sync animations of the procedure simulation with motion data of a mobile electronics device displaying the AR rendering to control playback of animations by changing the orientation of the mobile electronics device. The AR engine 1020 may include AR parameters tuned using procedure information, patient measurement data, and patient identifying information.

In summary, an example workflow for using an augmented reality system to generate a simulation for a cosmetic or reconstructive procedure involves a user (e.g. a patient or a physician) selecting a desired procedure from the procedures database 1002. Patient identifying information and measurements are then input manually, retrieved from the measurements database 1001 or are extracted from photos or 3D scans of the patient's body. The user selects the product(s) that will be used in the procedure and the artificial intelligence system 1003 tunes the modelling parameters, simulation parameters, and AR parameters based on, for example, the patient's demographics and physician's post-operative results. The 3D modelling engine 1010 then ingests the procedure information, patient identifying information and measurements, and the tuned modelling parameters to generate a 3D model. The simulation engine 1015 then ingests the procedure information, patient identifying information and measurements, the generated 3D model, and the tuned simulation parameters to generate a 3D model simulation. The AR engine 1020 then ingests the procedure information, patient identifying information and measurements, the generated 3D model simulation and the tuned AR parameters to generate an AR environment including one or more a 3D model objects. Next, the user streams live video of a body part selected for the procedure. The AR engine 1020 recognises the body part in the live video, tracks movements of the body part in real time, and pushes the matching 3D model object to the AR environment running on the user's device. Finally, the rendering logic 1050 renders the 3D model object within the AR environment over the body part in the live video. This provides an AR simulation that allows the user to view the 3D model object within the AR environment form multiple perspectives through an intuitive process. Where the user is the patient, the intuitive process may involve simply moving the actual body part in order to manipulate the position of the 3D model object in the AR environment.

1. A computer-implemented method of simulating the effect on a patient's body of a procedure, comprising:
receiving a selection of a procedure;
creating a pre-procedure 3D model of at least a part of the patient's body that would be affected by the procedure;
simulating the effects of the procedure on the patient's body and generating a plurality of post-procedure 3D models from the pre-procedure 3D model, each post-procedure 3D model representing the patient's body at a different time following the procedure; and
displaying any of the pre-procedure 3D model and the post-procedure 3D models over a still image or a video of the patient.

2. The method of claim 1 wherein the procedure comprises one of a cosmetic procedure, a reconstructive procedure, bariatric surgery, and implementation of a diet and/or a physical fitness plan.

3. The method of claim 1 further comprising:
receiving a selection of a potential complication of the procedure; and
simulating the effects of the complication on the patient's body and generating a plurality of post-complication 3D models from either the pre-procedure 3D model or a post-procedure 3D model, each post-complication 3D model representing the patient's body at a different time following the complication.

4. The method of claim 1 further comprising training a machine learning system on a training dataset comprising a plurality of 3D models of at least parts of a plurality of patients' bodies at different times following a procedure and using the machine learning system to simulate the effects of the procedure on the patient's body.

5. The method of claim 4 further comprising, following completion of the procedure, creating at least one 3D model of at least a part of the patient's body that has been affected by the procedure at at least one different time following the procedure and adding the at least one 3D model to the training dataset of the machine learning system.

6. The method of claim 1 wherein the post-procedure 3D models include a model representing the patient's body immediately after the procedure is completed, and at least one model representing the patient's body at a selected time during the procedure.

7. The method of claim 1 wherein the post-procedure 3D models include a model representing the patient's body immediately after the procedure, a model representing the patient's body after full recovery from the procedure, and at least one model representing the patient's body at a selected intervening time.

8. The method of claim 1 further comprising placing any of the pre-procedure 3D model and the post-procedure 3D models over a live video of the patient for displaying in an augmented reality environment.

9-32. (canceled)

33. The method of claim 1 further comprising training a machine learning system on training data comprising the effects of the procedure on a plurality of different patients' bodies, as performed by a plurality of different physicians, to generate a plurality of predictive models;
wherein simulating the effects of the procedure on the patient's body further comprises:
using a first predictive model of the plurality of predictive models, generating a first post-procedure 3D model of the at least part of the patient's body following the procedure, the first post-procedure 3D model simulating the effects of the procedure as performed by a first physician; and
using a second predictive model of the plurality of predictive models, generating a second post-procedure 3D model of the at least part of the patient's body following the procedure, the second post-procedure 3D model simulating the effects of the procedure as performed by a second physician.

34. A system for simulating the effect on a patient's body of a procedure, comprising:
a computer processor for receiving a selection of a procedure via a user interface;
a 3D modelling engine for creating a pre-procedure 3D model of at least a part of the patient's body that would be affected by the procedure;
a simulation engine for simulating the effects of the procedure on the patient's body and generating a plurality of post-procedure 3D models from the pre-procedure 3D model, each post-procedure 3D model representing the patient's body at a different time following the procedure; and
rendering logic for displaying, on a display device, any of the pre-procedure 3D model and the post-procedure 3D models over a still image or a video of the patient.

35. The system of claim 34 wherein the procedure comprises one of a cosmetic procedure, a reconstructive procedure, bariatric surgery, and implementation of a diet and/or a physical fitness plan.

36. The system of claim 34 wherein, responsive to the computer processor receiving a selection of a potential complication of the procedure via a user interface, the simulation engine simulates the effects of the complication on the patient's body and generates a plurality of post-complication 3D models from either the pre-procedure 3D model or a post-procedure 3D model, each post-complication 3D model representing the patient's body at a different time following the complication.

37. The system of claim 34 further comprising an artificial intelligence (AI) system, the AI system comprising a machine learning system, wherein the machine learning system is trained on a training dataset comprising a plurality of 3D models of at least parts of a plurality of patients' bodies at different times following a procedure and using the machine learning system to simulate the effects of the procedure on the patient's body.

38. The system of claim 37 wherein, following completion of the procedure, the 3D modelling engine creates at least one 3D model of at least a part of the patient's body that has been affected by the procedure at at least one different time following the procedure, and the at least one 3D model is added to the training dataset of the machine learning system.

39. The system of claim 34 wherein the post-procedure 3D models include a model representing the patient's body immediately after the procedure is completed, and at least one model representing the patient's body at a selected time during the procedure.

40. The system of claim 34 wherein the post-procedure 3D models include a model representing the patient's body immediately after the procedure, a model representing the patient's body after full recovery from the procedure, and at least one model representing the patient's body at a selected intervening time.

41. The system of claim 34 further comprising an augmented reality (AR) engine for placing any of the pre-procedure 3D model and the post-procedure 3D models over a live video of the patient for display by the rendering logic on the display device.

42. The system of claim 34 further comprising an artificial intelligence (AI) system, the AI system comprising a machine learning system, wherein the machine learning system is trained on training data comprising the effects of the procedure on a plurality of different patients' bodies, as performed by a plurality of different physicians, to generate a plurality of predictive models;
and wherein the simulation engine is further for simulating the effects of the procedure on the patient's body by:
(a) using a first predictive model of the plurality of predictive models, generating a first post-procedure 3D model of the at least part of the patient's body following the procedure, the first post-procedure 3D model simulating the effects of the procedure as performed by a first physician; and
(b) using a second predictive model of the plurality of predictive models, generating a second post-procedure 3D model of the at least part of the patient's body following the procedure, the second post-procedure 3D model simulating the effects of the procedure as performed by a second physician.

43. A computer-implemented method of simulating the effects of a medical procedure on a patient's body comprising:
training a machine learning system on training data comprising the effects of the medical procedure on a plurality of patients' bodies, as performed by a plurality of different physicians, to generate a plurality of predictive models;
creating a 3D model of at least a part of the patient's body that would be affected by the procedure;
using a first predictive model of the plurality of predictive models, generating a first modified 3D model of the at least part of the patient's body following the procedure that simulates the effects of the procedure as performed by a first physician; and
using a second predictive model of the plurality of predictive models, generating a second modified 3D model of the at least part of the patient's body following the procedure that simulates the effects of the procedure as performed by a second physician to obtain a virtual second opinion.
